(19) United States
(12) Patent Application Publication
(10) Pub. No.: US 2023/0416250 A1
(43) Pub. Date: Dec. 28, 2023

(54) METHODS OF PREPARING HETEROARYL-KETONE FUSED AZADECALIN GLUCOCORTICOID RECEPTOR MODULATORS

(71) Applicant: CORCEPT THERAPEUTICS INCORPORATED, MENLO PARK, CA (US)

(72) Inventors: Hazel Joan Hunt, Menlo Park, CA (US); Gary Patrick Reid, Menlo Park, CA (US); Jeffrey Mark Dener, Menlo Park, CA (US); Adam Daisuke Gammack Yamagata, Menlo Park, CA (US); Gareth C. Sheppard, Menlo Park, CA (US)

(21) Appl. No.: 18/342,239

(22) Filed: Jun. 27, 2023

Related U.S. Application Data
(60) Provisional application No. 63/367,151, filed on Jun. 28, 2022.

(51) Int. Cl. C07D 471/04

(52) U.S. Cl. CPC C07D 471/04; C07B 2200/13

(57) ABSTRACT

The present invention provides methods of preparing heteroaryl-ketone fused azadecalin glucocorticoid receptor modulators, and compositions having low impurity levels.

### CROSS-REFERENCES TO RELATED APPLICATIONS

This application claims priority to U.S. Provisional Application No. 63/367,151, filed Jun. 28, 2022, which is incorporated herein in its entirety for all purposes.

### BACKGROUND

There are two types of high-affinity receptors for corticosteroids; the type I (mineralocorticoid receptor, MR) and the type II (glucocorticoid receptor (GR), or cortisol receptor, GR). In most species, including man, the physiological glucocorticoid is cortisol (hydrocortisone). Glucocorticoids are secreted in response to ACTH (corticotropin), which shows both circadian rhythm variation and elevations in response to stress and food. Cortisol levels are responsive within minutes to many physical and psychological stresses, including trauma, surgery, exercise, anxiety and depression. Cortisol is a steroid and acts by binding to an intracellular, glucocorticoid receptor (GR). In man, glucocorticoid receptors are present in two forms: a ligand-binding GR-alpha of 777 amino acids; and, a GR-beta isoform which lacks the 50 carboxy terminal residues. Since these include the ligand binding domain, GR-beta is unable to bind ligand, is constitutively localized in the nucleus, and is transcriptionally inactive. The GR is also known as the GR-II.

The biologic effects of cortisol, including those caused by hypercortisolemia, can be modulated at the GR level using receptor modulators, such as agonists, partial agonists and antagonists. Several different classes of agents are able to block the physiologic effects of GR-agonist binding. These antagonists include compositions which, by binding to GR, inhibit the ability of an agonist to effectively bind to and/or activate the GR. One such known GR antagonist, mifepristone, has been found to be an effective anti-glucocorticoid agent in humans (Bertagna (1984) J. Clin. Endocrinol. Metab. 59:25). Mifepristone binds to the GR with high affinity, with a dissociation constant (K_{d}) of 10-9 M (Cadepond (1997) Annu. Rev. Med. 48:129). Dazucorilant (CORTI13176) is another such glucocorticoid receptor modulator compound, and has been described previously in PCT Publication No. WO 2013/177559, and U.S. Pat. No. 8,859,774. What is needed in the art are new methods of preparing dazucorilant having lower impurity content. Surprisingly, the present invention meets these and other needs.

### BRIEF SUMMARY OF THE INVENTION

In some embodiments, the present invention provides a method of preparing a compound of Formula I:

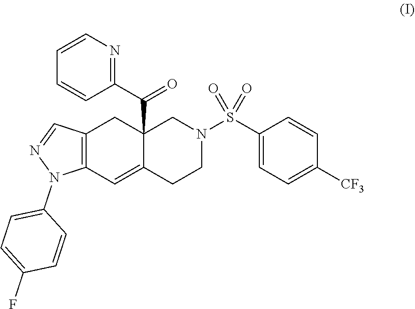 [SMILES: O=C(c1ccccn1)[C@]12Cc3cnn(-c4ccc(F)cc4)c3C=C1CCN(S(=O)(=O)c1ccc(C(F)(F)F)cc1)C2]

or a pharmaceutically acceptable salt thereof, comprising:
(a) forming a first reaction mixture comprising a compound of Formula II:

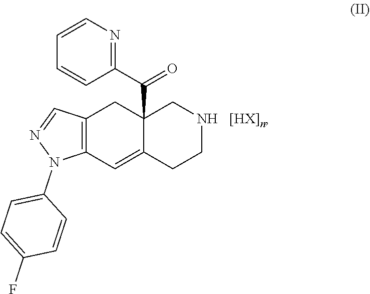

and
   4-(trifluoromethyl)benzenesulfonyl chloride:

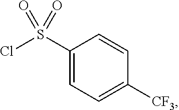 [SMILES: O=S(=O)(Cl)c1ccc(C(F)(F)F)cc1]

to prepare the compound of Formula I in a yield of at least 60% and a purity of at least 98%,
wherein
   HX is an acid solvate; and
   subscript n is 1 to 4.

In some embodiments, the present invention provides a method of preparing a compound of Formula I:

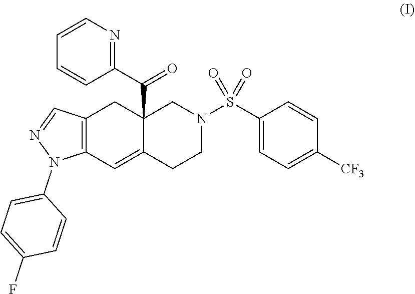 [SMILES: O=C(c1ccccn1)[C@]12Cc3cnn(-c4ccc(F)cc4)c3C=C1CCN(S(=O)(=O)c1ccc(C(F)(F)F)cc1)C2]

or a pharmaceutically acceptable salt thereof, comprises:
(a) forming a first reaction mixture comprising a compound of Formula IIa:

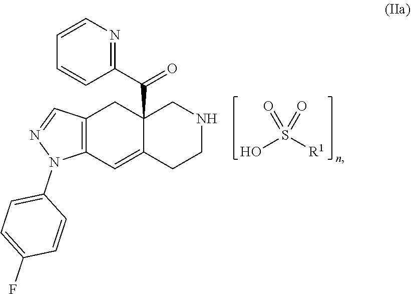

and
   4-(trifluoromethyl)benzenesulfonyl chloride:

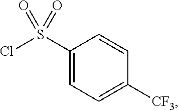 [SMILES: O=S(=O)(Cl)c1ccc(C(F)(F)F)cc1]

to prepare the compound of Formula I in a yield of at least 60% and a purity of at least 98%,
wherein
   R¹ is C₁₋₆ alkyl, C₁₋₁₀ haloalkyl, phenyl, or 4-methylphenyl; and subscript n is 1 to 4.

In some embodiments, the present invention provides a method of preparing a compound of Formula IIb:

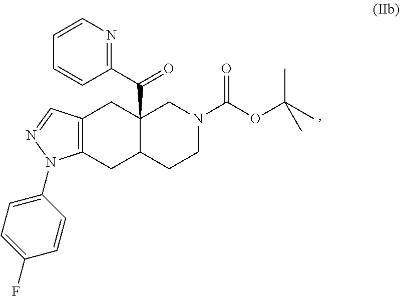 [SMILES: CC(C)(C)OC(=O)N1CCC2Cc3c(cnn3-c3ccc(F)cc3)C[C@]2(C(=O)c2ccccn2)C1]

comprising the step of:
(c) forming a third reaction mixture comprising a Grignard reagent, a compound of Formula III:

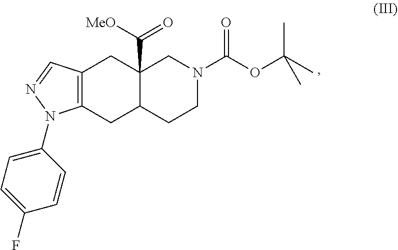 [SMILES: COC(=O)[C@]12Cc3cnn(-c4ccc(F)cc4)c3CC1CCN(C(=O)OC(C)(C)C)C2]

and
   2-bromo-pyridine:

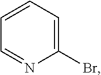

wherein the pyridine is present in a molar ratio of 2.8 to 3.2 to the compound of Formula III, and wherein the Grignard reagent is present in a molar ratio of 2.8 to 3.3 to the compound of Formula III, to prepare the compound of Formula IIb.

In some embodiments, the present invention provides a composition comprising:
a compound of Formula I in an amount of at least 99%:

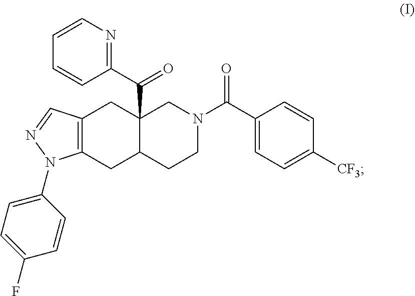 [SMILES: O=C(c1ccc(C(F)(F)F)cc1)N1CCC2Cc3c(cnn3-c3ccc(F)cc3)C[C@]2(C(=O)c2ccccn2)C1]

and
one or more impurity in an amount of from 0.01 to 1%.

In some embodiments, the present invention provides a crystalline form (R)-(1-(4-fluorophenyl)-1,4,5,6,7,8-hexahydro-4aH-pyrazolo[3,4-g]isoquinolin-4a-yl)(pyridin-2-yl)methanone methanesulfonic acid:

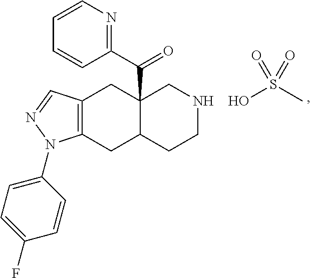

characterized by an X-ray powder diffraction (XRPD pattern having peaks at about 7.8, 14.6, and 15.5° 2-θ±0.2° 2-θ.

In some embodiments, the present invention provides a pharmaceutical composition comprising a low impurity composition of the present invention and a pharmaceutically acceptable excipient.

In some embodiments, the present invention provides a method of treating a disorder or condition through modulating a glucocorticoid receptor, the method comprising administering to a subject in need of such treatment, a therapeutically effective amount of a low impurity composition of the present invention, or a pharmaceutical composition of the present invention, thereby treating the disorder or condition.

In some embodiments, the present invention provides a method of treating a disorder or condition through antagonizing a glucocorticoid receptor, the method comprising administering to a subject in need of such treatment, a therapeutically effective amount of a low impurity composition of the present invention, or a pharmaceutical composition of the present invention.

In some embodiments, the present invention provides a method of treating amyotrophic lateral sclerosis (ALS), comprising administering to a subject in need thereof, a therapeutically effective amount of a low impurity composition of the present invention, or a pharmaceutical composition of the present invention, thereby treating fatty liver disease.

### BRIEF DESCRIPTION OF THE DRAWINGS

FIG. 1 shows the synthetic scheme for preparing (R)-(1-(4-fluorophenyl)-6-((4-(trifluoromethyl)phenyl)sulfonyl)-4,4a,5,6,7,8-hexahydro-1H-pyrazolo[3,4-g]isoquinolin-4a-yl)(pyridin-2-yl)methanone according to Example 1.

FIG. 2 shows the X-ray diffraction pattern (XRPD) for the crystalline compound (R)-1-(4-fluorophenyl)-4a-picolinoyl-4,4a,5,6,7,8-hexahydro-1H-pyrazolo[3,4-g]isoquinolin-6-ium methanesulfonate.

FIG. 3 shows the shows the differential scanning calorimetry (DSC) thermogram for the crystalline compound (R)-1-(4-fluorophenyl)-4a-picolinoyl-4,4a,5,6,7,8-hexahydro-1H-pyrazolo[3,4-g]isoquinolin-6-ium methanesulfonate.

FIG. 4 shows the shows the thermal gravimetric analysis (TGA) for the crystalline compound (R)-1-(4-fluorophenyl)-4a-picolinoyl-4,4a,5,6,7,8-hexahydro-1H-pyrazolo[3,4-g]isoquinolin-6-ium methanesulfonate.

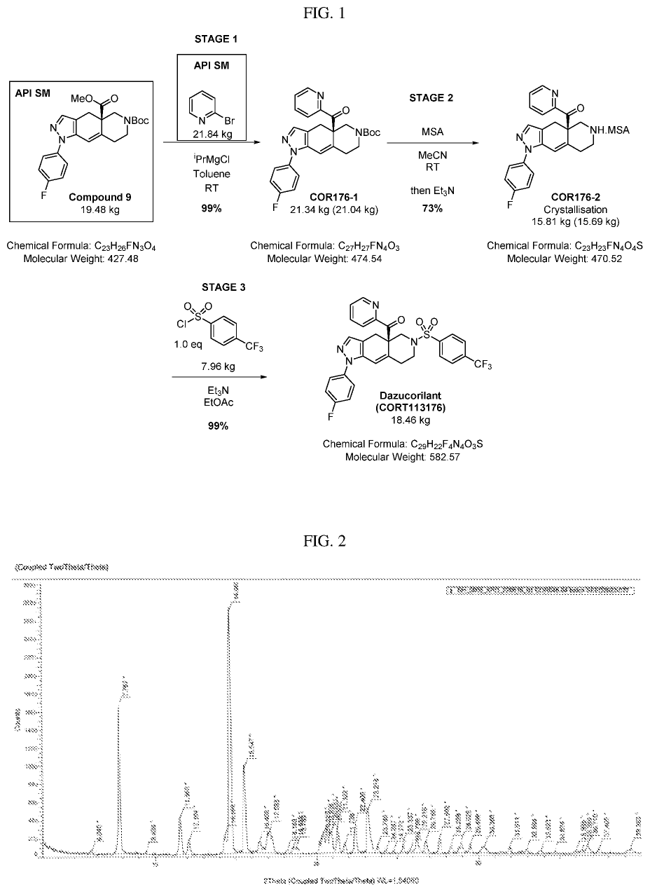

### DETAILED DESCRIPTION OF THE INVENTION

### I. General

The instant disclosure describes new methods of preparing the compound of Formula I, (R)-(1-(4-fluorophenyl)-6-((4-(trifluoromethyl)phenyl)sulfonyl)-4,4a,5,6,7,8-hexahydro-1H-pyrazolo[3,4-g]isoquinolin-4a-yl)(pyridin-2-yl)methanone (dazucorilant), having lower impurity levels than the methods previously described. The new methods of preparing dazucorilant have improved safety and cost effectiveness, and can be prepared at larger scale, compared to known methods. Dazucorilant can be prepared as in Example 1 of U.S. Pat. No. 8,859,774. The instant disclosure also describes compositions of dazucorilant that have lower impurity levels.

### II. Definitions

“About” when referring to a value includes the stated value+/−10% of the stated value. For example, about 50% includes a range of from 45% to 55%, while about 20 molar equivalents includes a range of from 18 to 22 molar equivalents. Accordingly, when referring to a range, “about” refers to each of the stated values+/−10% of the stated value of each end of the range. For instance, a ratio of from about 1 to about 3 (weight/weight) includes a range of from 0.9 to 3.3.

“Forming a reaction mixture” refers to the process of bringing into contact at least two distinct species such that they mix together and can react. It should be appreciated, however, the resulting reaction product can be produced directly from a reaction between the added reagents or from an intermediate from one or more of the added reagents which can be produced in the reaction mixture.

“Dissolve”, “dissolving” or “dissolution” refers to a solid material that is substantially soluble in a particular solvent. For example, the solid material can be greater than 90% soluble in the solvent, or greater than 91, 92, 93, 94, 95, 96, 97, 98, or greater than 99% soluble in the solvent.

“Distilling”, “distill” or “distillation” refers to the separation of components in a liquid mixture using a combination of temperature and pressure. The target component is converted from a liquid to a gas followed by condensing the gas back to a liquid to separate the target component from the other components of the mixture.

“Eluting”, “elute” or “elution” refers to the process of separating a target component from other components in a mixture by passing the mixture over a stationary phase. The target component is eluted from the stationary phase using a mobile phase that can include any suitable solvent or acid.

“Concentrate” or “concentrating” refers to the process of removing solvent or diluent from a mixture to increase the molar concentration of a component in the mixture.

Concentrating can be accomplished by a variety of methods such as distillation or rotary evaporation. Concentrating can include removing some or all of the solvent or diluent.

“Precipitate”, “precipitated” or “precipitation” refers to a solid formed from a solution, such as adding a first solvent in which a compound is dissolved to an excess of a second solvent in which the compound is not substantially soluble such that the dissolved compound comes out of solution and forms a solid.

“Substantially free” refers to a composition having an undesired component in an amount less than 5%, less than 1%, less than 0.5% or even less than 0.1% by HPLC peak area.

“Aqueous phase” refers to a mixture containing water.

“Organic phase” refers to a mixture containing water-miscible or -immiscible solvents capable of dissolving either or both of water-soluble and water-insoluble organic compounds. The organic phase of the present invention can formed from one or more organic solvents. Exemplary organic solvents can be non-polar aprotic solvents, polar aprotic solvents, and polar protic solvents. Representative solvents include, but are not limited to, pentanes, hexanes, hexane, heptanes, benzene, toluene, diethyl ether, tetrahydrofuran, acetone, ethyl acetate, acetonitrile, methylene chloride, chloroform, etc.

“Acid” refers to a compound that is capable of donating a proton (H⁺) under the Bronsted-Lowry definition, or is an electron pair acceptor under the Lewis definition. Acids useful in the present invention are Bronsted-Lowry acids that include, but are not limited to, alkanoic acids or carboxylic acids (formic acid, acetic acid, citric acid, lactic acid, oxalic acid, etc.), sulfonic acids and mineral acids, as defined herein. Mineral acids are inorganic acids such as hydrogen halides (hydrofluoric acid, hydrochloric acid, hydrobromic acid, etc.), halogen oxoacids (hypochlorous acid, perchloric acid, etc.), as well as sulfuric acid, nitric acid, phosphoric acid, chromic acid and boric acid. Sulfonic acids include methanesulfonic acid, benzenesulfonic acid, p-toluenesulfonic acid, trifluoromethanesulfonic acid, camphorsulfonic acid, among others.

“Grignard reagent” refers to a reagent containing a complex of magnesium metal, a halide, and an alkyl ligand, capable of forming a carbon-carbon bond. Representative Grignard reagents include, but are not limited to, iPrMgCl and iPrMgBr.

“Non-nucleophilic base” refers to a base that is a moderate to strong base but at the same time is a poor nucleophile. Representative non-nucleophilic bases include bases such as potassium carbonate, sodium carbonate, potassium tert-butoxide, and sodium tert-butoxide, as well as amine bases, such as triethylamine (Et₃N), N,N-diisopropylethylamine (iPr₂NEt; DIPEA), N,N-diethylaniline, pyridine, 2,6-lutidine, 2,4,6-collidine, 4-dimethylaminopyridine, and quinuclidine. Non-nucleophilic base includes non-nucleophilic amine bases.

“Solvent” refers to a substance, such as a liquid, capable of dissolving a solute. Solvents can be polar or non-polar, protic or aprotic. Polar solvents typically have a dielectric constant greater than about 5 or a dipole moment above about 1.0, and non-polar solvents have a dielectric constant below about 5 or a dipole moment below about 1.0. Protic solvents are characterized by having a proton available for removal, such as by having a hydroxy or carboxy group. Aprotic solvents lack such a group. Representative polar protic solvents include alcohols (methanol, ethanol, propanol, isopropanol, etc.), acids (formic acid, acetic acid, etc.) and water. Representative polar aprotic solvents include dichloromethane, chloroform, tetrahydrofuran, diethyl ether, 1,4-dioxane, acetone, ethyl acetate, dimethylformamide, dimethylacetamide, acetonitrile and dimethyl sulfoxide. Representative non-polar solvents include alkanes (pentanes, hexanes, etc.), cycloalkanes (cyclopentane, cyclohexane, etc.), benzene, and toluene. Other solvents are useful in the present invention.

“Room temperature” is the range of air temperatures generally considered to be suitable for human occupancy, or between about 15 degrees Celsius (59 degrees Fahrenheit) and 25 degrees Celsius (77 degrees Fahrenheit).

“Vacuum” or “reduced pressure” refers to a pressure that is less than atmospheric pressure. Atmospheric pressure is measured as about 1013 mbar, 760 mm Hg, or about 14.7 psi. Accordingly, vacuum can be less than 1013 mbar, or less than 100, 10, 1, 0.1, or less than 0.01 mbar.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Halogen” refers to fluorine, chlorine, bromine and iodine.

“Haloalkyl” refers to alkyl, as defined above, where some or all of the hydrogen atoms are replaced with halogen atoms. As for alkyl groups, haloalkyl groups can have any suitable number of carbon atoms, such as C₁₋₆. For example, haloalkyl includes trifluoromethyl, fluoromethyl, etc. The term “perfluoro” can be used to define a compound or radical where all the hydrogens are replaced with fluorine. For example, perfluoromethyl refers to 1,1,1-trifluoromethyl.

“Pharmaceutically acceptable salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid, methane sulfonic acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Composition” as used herein is intended to encompass a product comprising the specified ingredients in the specified amounts, as well as any product, which results, directly or indirectly, from combination of the specified ingredients in the specified amounts. By “pharmaceutically acceptable” it is meant the carrier(s), diluent(s) or excipient(s) must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present invention include, but are not limited to, binders, fillers, disintegrants, lubricants, surfactants, coatings, sweeteners, flavors and colors. One of skill in the art will recognize that other pharmaceutical excipients are useful in the present invention.

“Treat”, “treating” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Patient” or “subject” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, horse, and other non-mammalian animals. In some embodiments, the patient is human.

“Therapeutically effective amount” refers to an amount of a compound or of a pharmaceutical composition useful for treating or ameliorating an identified disease or condition, or for exhibiting a detectable therapeutic or inhibitory effect. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Glucocorticoid receptor” (“GR”) refers to one of the family of intracellular receptors which specifically bind to cortisol and/or cortisol analogs such as dexamethasone (See, e.g., Turner & Muller, J. Mol. Endocrinol. Oct. 1, 2005 35 283-292). The glucocorticoid receptor is also referred to as the cortisol receptor. The term includes isoforms of GR, recombinant GR and mutated GR.

A cortisol receptor is a glucocorticoid receptor (GR), specifically the type II GR, which specifically binds cortisol and/or cortisol analogs such as dexamethasone (See, e.g., Turner & Muller, J. Mol. Endocrinol. Oct. 1, 2005 35 283-292).

“Mineralocorticoid receptor” (MR) refers to a type I glucocorticoid receptor (GR I), which is activated by aldosterone in humans.

“Glucocorticoid receptor modulator” (GRM) refers to any compound which modulates any biological response associated with the binding of a glucocorticoid receptor to an agonist. As used herein, with respect to a GRM, the glucocorticoid receptor may be GR, or both. For example, a GRM that acts as an agonist, such as dexamethasone, increases the activity of tyrosine aminotransferase (TAT) in HepG2 cells (a human liver hepatocellular carcinoma cell line; ECACC, UK). A GRM that acts as an antagonist, such as mifepristone, inhibits the agonist-induced increase in the activity of tyrosine aminotransferase (TAT) in HepG2 cells. TAT activity can be measured as outlined in the literature by A. Ali et al., J. Med. Chem., 2004, 47, 2441-2452.

“Glucocorticoid receptor antagonist” (GRA) refers to any compound which inhibits any biological response associated with the binding of a glucocorticoid receptor to an agonist. As used herein, with respect to a GRA, the glucocorticoid receptor may be GR, or both. Accordingly, GR antagonists can be identified by measuring the ability of a compound to inhibit the effect of dexamethasone. TAT activity can be measured as outlined in the literature by A. Ali et al., J. Med. Chem., 2004, 47, 2441-2452. An inhibitor is a compound with an IC₅₀ (half maximal inhibition concentration) of less than 10 micromolar. See Example 1 of U.S. Pat. No. 8,685,973, the entire contents of which is hereby incorporated by reference in its entirety.

“Modulate” and “modulating” are used in accordance with its plain ordinary meaning and refer to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Modulator” refers to a composition that increases or decreases the level of a target molecule or the function of a target molecule or the physical state of the target of the molecule.

“Antagonize’ and “antagonizing” refer to inhibiting the binding of an agonist at a receptor molecule or to inhibiting the signal produced by a receptor-agonist. A receptor antagonist inhibits or dampens agonist-mediated responses, such as gene expression.

“Antagonist” refers to a substance capable of detectably lowering expression or activity of a given gene or protein. The antagonist can inhibit expression or activity 10%, 20%, 30%, 40%, 50%, 60%, 70%, 80%, 90%, 100% or less in comparison to a control in the absence of the antagonist. In some embodiments, the inhibition is 1.5-fold, 2-fold, 3-fold, 4-fold, 5-fold, 10-fold, or more than the expression or activity in the absence of the antagonist.

“Inhibition”, “inhibits” and “inhibitor” refer to a compound that prohibits or a method of prohibiting, a specific action or function.

“Disorder” or “condition” refers to a state of being or health status of a patient or subject capable of being treated with the glucocorticoid receptor modulator of the present invention. In some embodiments, examples of disorders or conditions include, but are not limited to, amyotrophic lateral sclerosis (ALS).

### III. Method of Preparing Formula I from Formula IIa

In some embodiments, the present invention provides a method of preparing a compound of Formula I:

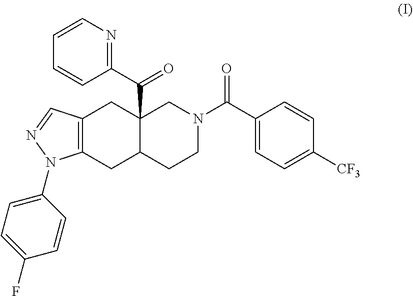 [SMILES: O=C(c1ccc(C(F)(F)F)cc1)N1CCC2Cc3c(cnn3-c3ccc(F)cc3)C[C@]2(C(=O)c2ccccn2)C1]

or a pharmaceutically acceptable salt thereof, comprising:
(a) forming a first reaction mixture comprising a compound of Formula II:

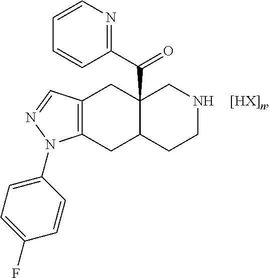

and
   4-(trifluoromethyl)benzenesulfonyl chloride:

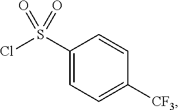 [SMILES: O=S(=O)(Cl)c1ccc(C(F)(F)F)cc1]

to prepare the compound of Formula I in a yield of at least 60% and a purity of at least 98%,
wherein HX is an acid solvate; and subscript n is 1 to 4.

In some embodiments, HX is

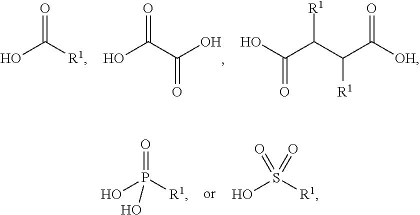

wherein R¹ is C₁₋₆ alkyl, C₁₋₁₀ haloalkyl, phenyl, 4-methylphenyl, 4-NO₂-phenyl, —OC(O)-phenyl or

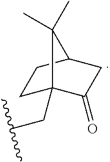 [SMILES: *CC12CCC(CC1=O)C2(C)C]

In some embodiments, HX is

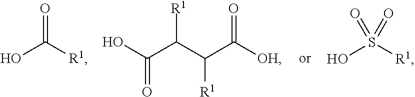

wherein R¹ is C₁₋₆ alkyl, phenyl, 4-methylphenyl, 4-NO₂-phenyl, —OC(O)-phenyl or

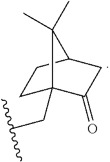 [SMILES: *CC12CCC(CC1=O)C2(C)C]

In some embodiments, HX is

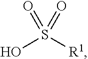

wherein R¹ is C₁₋₆ alkyl, phenyl, 4-methylphenyl, 4-NO₂-phenyl, —OC(O)-phenyl or

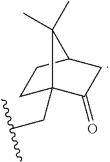 [SMILES: *CC12CCC(CC1=O)C2(C)C]

In some embodiments, HX is

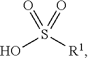

wherein R¹ is C₁₋₆ alkyl.

In some embodiments, HX is MeS(O)₂OH,

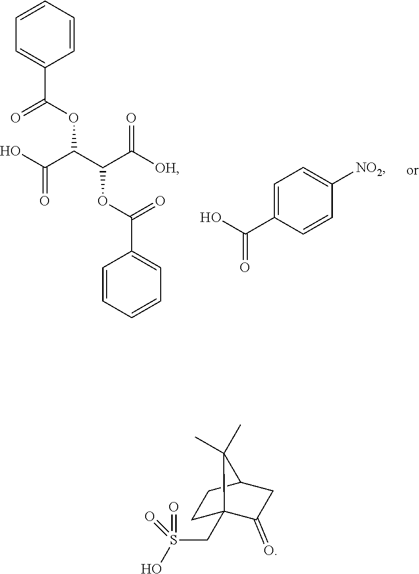

In some embodiments, HX is MeS(O)₂OH.

In some embodiments, the present invention provides a method for preparing the compound of Formula I:

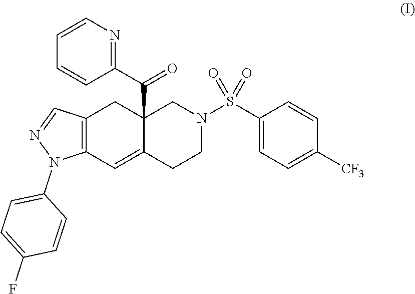 [SMILES: O=C(c1ccccn1)[C@]12Cc3cnn(-c4ccc(F)cc4)c3C=C1CCN(S(=O)(=O)c1ccc(C(F)(F)F)cc1)C2]

or a pharmaceutically acceptable salt thereof, comprises:
(a) forming a first reaction mixture comprising a compound of Formula IIa:

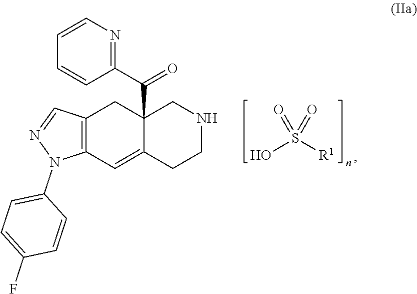

and
   4-(trifluoromethyl)benzenesulfonyl chloride:

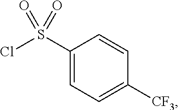 [SMILES: O=S(=O)(Cl)c1ccc(C(F)(F)F)cc1]

to prepare the compound of Formula I in a yield of at least 60% and a purity of at least 98%,
wherein R¹ is C₁₋₆ alkyl, C₁₋₁₀ haloalkyl, phenyl, or 4-methylphenyl; and subscript n is 1 to 4.

In some embodiments, the compound of Formula I contains less than 1% of the compound of Formula X-5:

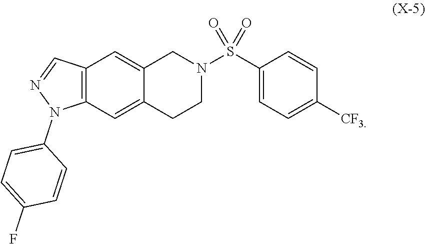 [SMILES: O=S(=O)(c1ccc(C(F)(F)F)cc1)N1CCc2cc3c(cnn3-c3ccc(F)cc3)cc2C1]

The compound of Formula I can be prepared from the compound of Formula IIa:

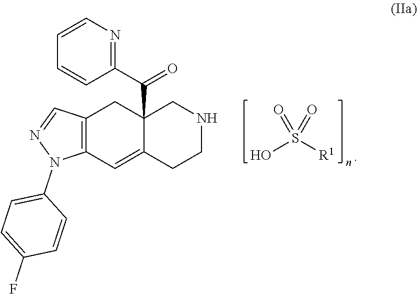

A. Preparing Formula I from Formula IIa

In some embodiments, the present invention provides a method of preparing a compound of Formula I:

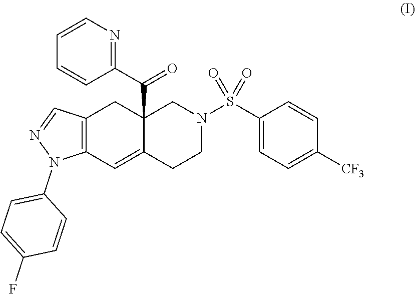 [SMILES: O=C(c1ccccn1)[C@]12Cc3cnn(-c4ccc(F)cc4)c3C=C1CCN(S(=O)(=O)c1ccc(C(F)(F)F)cc1)C2]

or a pharmaceutically acceptable salt thereof, comprising:
(a) forming a first reaction mixture comprising a compound of Formula IIa:

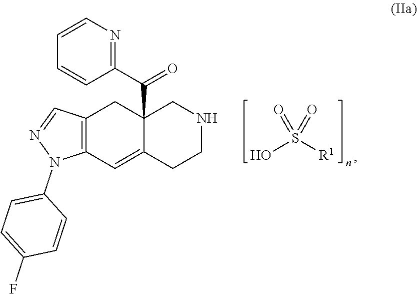

and
   4-(trifluoromethyl)benzenesulfonyl chloride:

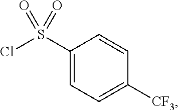 [SMILES: O=S(=O)(Cl)c1ccc(C(F)(F)F)cc1]

to prepare the compound of Formula I in a yield of at least 60% and a purity of at least 98%,
wherein
   R¹ is C₁₋₆ alkyl, C₁₋₁₀ haloalkyl, phenyl, or 4-methylphenyl; and subscript n is 1 to 4.

In some embodiments, the compound of Formula I is prepared in a yield of at least 75% and a purity of at least 98%.

In some embodiments, R¹ is C₁₋₂ alkyl, C₁₋₂ haloalkyl, phenyl, or 4-methylphenyl. In some embodiments, R¹ is methyl, ethyl, —CF₃, phenyl, or 4-methylphenyl. In some embodiments, R¹ is methyl.

Subscript n can be 1, 2, 3 or 4. In some embodiments, subscript n is 1. In some embodiments, subscript n is 2. In some embodiments, subscript n is 3. In some embodiments, subscript n is 4. In some embodiments, the compound of Formula IIa has the structure:

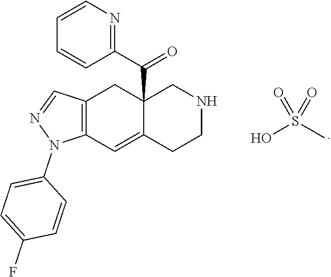

In some embodiments, the first reaction mixture further comprises a non-nucleophilic amine base. Any suitable non-nucleophilic amine base can be used in the first reaction mixture. In some embodiments, the non-nucleophilic amine base comprises trimethylamine, triethylamine (Et₃N), N,N-diisopropylethylamine (iPr₂NEt; DIPEA), N,N-dimethylisopropylamine (DIMPA), 1-ethylpiperidine, N-methylmorpholine, N-methylpyrrolidine, pyridine, N,N-dimethylaniline, N,N-diethylaniline, 2,6-lutidine, 2,4,6-collidine, 4-dimethyl aminopyridine (DMAP), quinuclidine, 4-pyrrolidinopyridine, 1,4-diazabicyclo[2.2.2]octane (DABCO), or mixtures thereof. In some embodiments, the non-nucleophilic amine base comprises triethylamine.

The first reaction mixture can include any suitable solvent. For example, the solvent can be an organic solvent including, but not limited to, ethyl acetate, isopropylacetate, and n-butyl acetate. In some embodiments, the first reaction mixture further comprises a first solvent. In some embodiments, the first solvent includes ethyl acetate, isopropyl acetate, n-butyl acetate, or mixtures thereof. In some embodiments, the first reaction mixture further comprises ethyl acetate.

The sulfonyl chloride used in the first reaction mixture can be present in any suitable molar ratio to the compound of Formula II or Formula IIa. For example, the sulfonyl chloride can be present in a molar ratio of from 0.5 to 2.0 to the compound of Formula II or Formula IIa, from 0.5 to 1.5, from 0.6 to 1.4, from 0.7 to 1.3, from 0.8 to 1.2, or from 0.9 to 1.1 to the compound of Formula II or Formula IIa. In some embodiments, the sulfonyl chloride is present in a molar ratio of 0.5 to 1.5 to the compound of Formula IIa. The sulfonyl chloride can be present in a molar ratio of 0.5, 0.6, 0.7, 0.8, 0.9, 1.0, 1.1, 1.2, 1.3, 1.4 or 1.5 to the compound of Formula II or Formula IIa. In some embodiments, the sulfonyl chloride can be present in a molar ratio of about 1.0 to the compound of Formula IIa.

The compound of Formula I can be prepared in any suitable yield. For example, the compound of Formula I can be prepared in a yield of at least 10, 20, 30, 40, 50, 60, 65, 70, 75, 80, 85, 90, or at least 95%. In some embodiments, the compound of Formula I can be prepared in a yield of at least 75%.

The compound of Formula I can be prepared in any suitable purity. For example, the compound of Formula I can be prepared in a purity of at least 90%, or 91, 92, 93, 94, 95, 96, 97, 98, or at least 99%. In some embodiments, the compound of Formula I can be prepared in a purity of at least 96%. In some embodiments, the compound of Formula I can be prepared in a purity of at least 97%. In some embodiments, the compound of Formula I can be prepared in a purity of at least 98%. In some embodiments, the compound of Formula I can be prepared in a purity of at least 99%.

In some embodiments, the method of preparing the compound of Formula I further includes the step of: (a1) adding an amino scavenging agent to the first reaction mixture to remove unreacted 4-(trifluoromethyl)benzenesulfonyl chloride. The amino scavenging agent can be any suitable amino compound capable of reacting with the sulfonyl chloride. In some embodiments, the amino scavenging agent comprises N-methylpiperazine, N¹,N¹-dimethylethane-1,2-diamine, N¹,N¹,N²-trimethylethane-1,2-diamine, or N¹,N¹-bis(2-aminoethyl)ethane-1,2-diamine. In some embodiments, the amino scavenging agent includes N-methylpiperazine. In some embodiments, the amino scavenging agent is N-methylpiperazine.

The amino scavenging agent can be present in any suitable amount. For example, the amino scavenging agent can be present in a molar ratio of less than 1.0 to the compound of Formula II or Formula IIa, or less than 0.9, 0.8, 0.75, 0.7, 0.6, 0.5, 0.4, 0.3, or less than 0.25 to the compound of Formula II or Formula IIa. In some embodiments, the amino scavenging agent is present in a molar ratio of about 0.25 to the compound of Formula IIa.

In some embodiments, the method of preparing the compound of Formula I further includes the steps of: (a2) adding a first acid and water to the first reaction mixture to partition the first reaction mixture into the first water mixture and the first organic mixture; and (a3) separating the first water mixture from the first organic mixture.

The first acid can include any suitable acid. In some embodiments, the first acid includes hydrochloric acid.

In some embodiments, the method of preparing the compound of Formula I further comprises the steps of:
(a4) concentrating the first organic mixture;
(a5) adding ethanol to the concentrated first organic mixture; and
(a6) adding water to the concentrated first organic mixture to precipitate the compound of Formula I.

In some embodiments, the method of preparing the compound of Formula I from the compound of Formula IIa comprises the steps of:
(a) forming the first reaction mixture comprising the compound of Formula IIa having the structure:

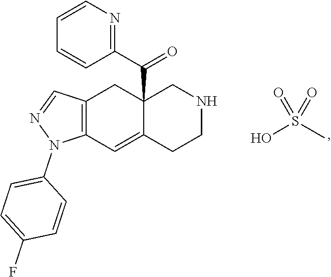

triethylamine, ethyl acetate, and 4-(trifluoromethyl)benzenesulfonyl chloride:

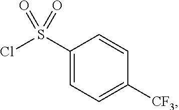 [SMILES: O=S(=O)(Cl)c1ccc(C(F)(F)F)cc1]

wherein the sulfonyl chloride is present in a molar ratio of about 1.0 to the compound of Formula IIa;
(a1) adding N-methylpiperazine to the first reaction mixture to remove unreacted 4-(trifluoromethyl)benzenesulfonyl chloride;
(a2) adding HCl and water to the first reaction mixture to partition the first reaction mixture into the first water mixture and the first organic mixture;
(a3) separating the first water mixture from the first organic mixture;
(a4) concentrating the first organic mixture;
(a5) adding ethanol to the concentrated first organic mixture; and
(a6) adding water to the concentrated first organic mixture to precipitate the compound of Formula I in a yield of at least 75% and a purity of at least 98%.

The compound of Formula I can be prepared with any acceptable amount of Formula X-5:

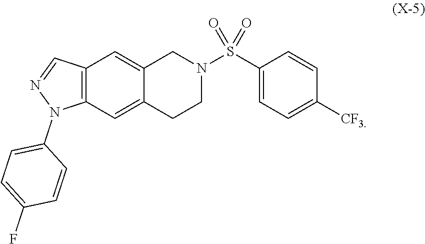 [SMILES: O=S(=O)(c1ccc(C(F)(F)F)cc1)N1CCc2cc3c(cnn3-c3ccc(F)cc3)cc2C1]

For example, the compound Formula I can be prepared containing less than 5%, or 4, 3, 2, 1, 0.9, 0.8, 0.75, 0.7, 0.6, 0.5, 0.4, 0.3, 0.25, 0.2, or less than 0.1% of the compound of Formula X-5. In some embodiments, the compound of Formula I can be prepared containing less than 1% of the compound of Formula X-5:

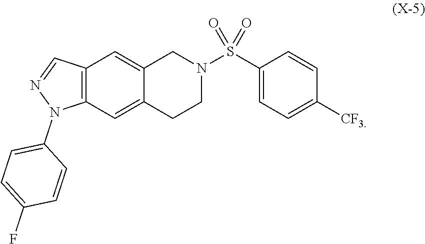 [SMILES: O=S(=O)(c1ccc(C(F)(F)F)cc1)N1CCc2cc3c(cnn3-c3ccc(F)cc3)cc2C1]

In some embodiments, the compound of Formula I can be prepared containing less than 0.75% of the compound of Formula X-5. In some embodiments, the compound of Formula I can be prepared containing less than 0.5% of the compound of Formula X-5. In some embodiments, the compound of Formula I can be prepared containing less than 0.1% of the compound of Formula X-5.

B. Preparing Formula IIa from Formula IIb

The compound of Formula IIa can be prepared from the compound of Formula IIb. In some embodiments, the present invention provides a method of preparing a compound of Formula IIa:

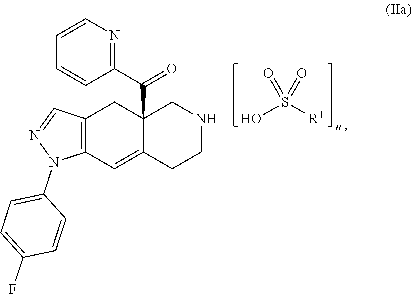

comprising:
(b) forming a second reaction mixture comprising a compound of Formula IIb:

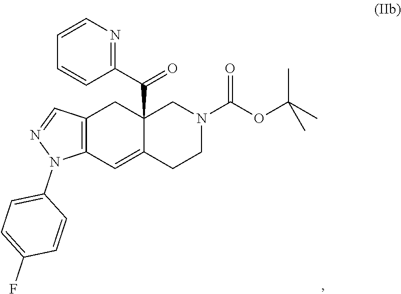 [SMILES: CC(C)(C)OC(=O)N1CCC2=Cc3c(cnn3-c3ccc(F)cc3)C[C@]2(C(=O)c2ccccn2)C1]

and
   a sulfonic acid of the formula:

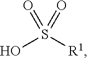

to form the compound of Formula IIa,
   wherein
R¹ is C₁₋₆ alkyl, C₁₋₁₀ haloalkyl, phenyl, or 4-methylphenyl; and subscript n is 1 to 4.

Subscript n can be 1, 2, 3 or 4. In some embodiments, subscript n is 1. In some embodiments, subscript n is 2. In some embodiments, subscript n is 3. In some embodiments, subscript n is 4. In some embodiments, the compound of Formula IIa has the structure:

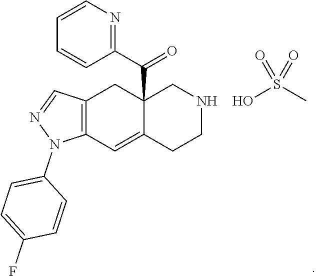

The second reaction mixture can include any suitable solvent. In some embodiments, the second reaction mixture includes a second solvent. The second solvent can include, but is not limited to, pentanes, hexanes, heptanes, benzene, toluene, diethyl ether, tetrahydrofuran, acetone, ethyl acetate, acetonitrile, methylene chloride, and chloroform. In some embodiments, the second solvent includes acetonitrile, cyclopentylmethyl ether (CPME), tetrahydrofuran, 2-methyltetrahydrofuran, dimethylsulfoxide (DMSO), N,N-dimethylformamide (DMF), methanol, ethanol, diethyl ether, methyl-t-butyl ether (MTBE), toluene, or combinations thereof. In some embodiments, the second reaction mixture includes acetonitrile.

C. Preparing Formula IIb from Formula III

The compound of Formula IIb can be prepared by a variety of methods. In some embodiments, the compound of Formula IIb can be prepared by the steps of:
(c) forming a third reaction mixture comprising a Grignard reagent, a compound of Formula III:

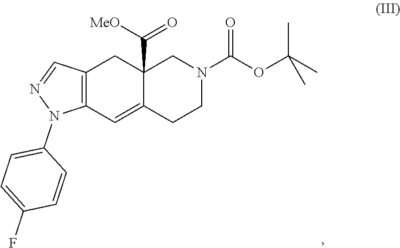 [SMILES: COC(=O)[C@]12Cc3cnn(-c4ccc(F)cc4)c3C=C1CCN(C(=O)OC(C)(C)C)C2]

2-bromo-pyridine:

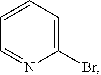

wherein the pyridine is present in a molar ratio of 2.8 to 3.2 to the compound of Formula III, and wherein the Grignard reagent is present in a molar ratio of 2.8 to 3.3 to the compound of Formula III, to prepare the compound of Formula IIb.

The Grignard reagent can be any suitable Grignard reagent. In some embodiments, the Grignard reagent comprises iPrMgCl or iPrMgBr. In some embodiments, the Grignard reagent comprises iPrMgCl.

The Grignard reagent can be present in any suitable molar ratio to the compound of Formula III. For example, the Grignard reagent can be present in a molar ratio of from 2.0 to 4.0, or 2.5 to 3.5, from 2.6 to 3.4, from 2.7 to 3.3, 2.8 to 3.3, 2.8 to 3.2, from 2.9 to 3.2, from 2.9 to 3.1 to the compound of Formula III. In some embodiments, the Grignard reagent can be present in a molar ratio of from 2.8 to 3.3 to the compound of Formula III. In some embodiments, the Grignard reagent can be present in a molar ratio of from 2.9 to 3.2 to the compound of Formula III. The Grignard reagent can be present in a molar ratio of about 2.90, or about 2.95, 3.00, 3.05, 3.10, 3.15, or about 3.20 to the compound of Formula III. In some embodiments, the Grignard reagent can be present in a molar ratio of about 3.0 to the compound of Formula III.

The pyridine can be present in any suitable ratio to the compound of Formula III. For example, the pyridine can be present in a molar ratio of 2.0 to 4.0, or 2.5 to 3.5, from 2.6 to 3.4, from 2.7 to 3.3, 2.8 to 3.2, from 2.9 to 3.1 to the compound of Formula III. In some embodiments, the pyridine can be present in a molar ratio of from 2.8 to 3.2 to the compound of Formula III. The pyridine can be present in a molar ratio of about 2.5, or about 2.6, 2.7, 2.8, 2.9, 3.0, 3.1, 3.2, 3.3, 3.4 or about 3.5 to the compound of Formula III. In some embodiments, the pyridine can be present in a molar ratio of about 3.0 to the compound of Formula III.

The third reaction mixture can also include a third solvent. The third solvent can be any suitable solvent including, but not limited to, tetrahydrofuran, 2-methyltetrahydrofuran, toluene, xylene, or combinations thereof. In some embodiments, the third reaction mixture further comprises a third solvent. In some embodiments, the third solvent can be tetrahydrofuran, 2-methyltetrahydrofuran, toluene, xylene, or combinations thereof. In some embodiments, the third reaction mixture further comprises 2-methyltetrahydrofuran and toluene.

In some embodiments, the method of preparing the compound of Formula IIb also includes: (c1) adding a third acid and water to the third reaction mixture to form a workup mixture; and (c2) distilling the workup mixture to form an intermediate mixture comprising the compound of Formula IIb.

The third acid of step (c1) can be any suitable acid. In some embodiments, the third acid comprises formic acid, acetic acid, propanoic acid, butyric acid, hexanoic acid, octanoic acid, trifluoroacetic acid, or mixtures thereof. In some embodiments, the third acid comprises acetic acid.

In some embodiments, the second reaction mixture further comprises the intermediate mixture comprising the compound of Formula IIb.

In some embodiments, the method of preparing the compound of Formula IIa comprises the steps of:
(c) forming the third reaction mixture comprising tetrahydrofuran, toluene, iPrMgCl, the compound of Formula III:

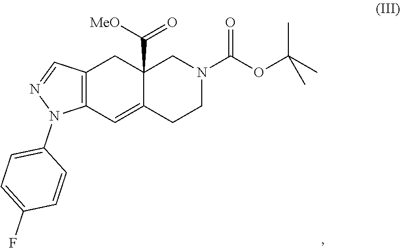 [SMILES: COC(=O)[C@]12Cc3cnn(-c4ccc(F)cc4)c3C=C1CCN(C(=O)OC(C)(C)C)C2]

and
   2-bromo-pyridine:

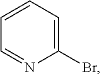

wherein the pyridine is present in the molar ratio of about 3.0 to the compound of Formula III, and wherein the Grignard reagent is present in the molar ratio of 3.0 to the compound of Formula III;
(c1) adding acetic acid and water to the third reaction mixture to form the workup mixture;
(c2) distilling the workup mixture to form the intermediate mixture comprising the compound of Formula IIb:

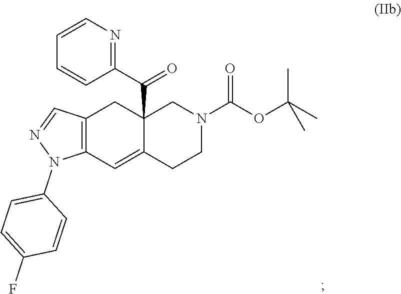 [SMILES: CC(C)(C)OC(=O)N1CCC2=Cc3c(cnn3-c3ccc(F)cc3)C[C@]2(C(=O)c2ccccn2)C1]

and
(b) forming the second reaction mixture comprising the intermediate mixture, acetonitrile, and methanesulfonic acid, to form the compound of Formula IIa having the structure:

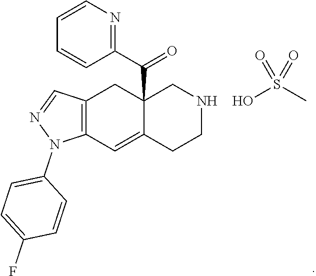

In some embodiments, the present invention provides a method of preparing a compound of Formula I:

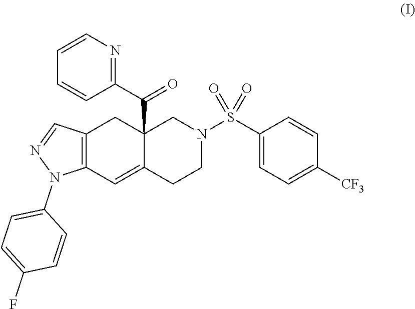 [SMILES: O=C(c1ccccn1)[C@]12Cc3cnn(-c4ccc(F)cc4)c3C=C1CCN(S(=O)(=O)c1ccc(C(F)(F)F)cc1)C2]

or a pharmaceutically acceptable salt thereof, comprising:
(c) forming a third reaction mixture comprising tetrahydrofuran, toluene, iPrMgCl, a compound of Formula III:

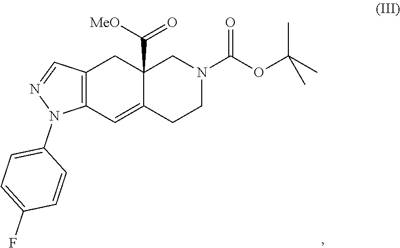 [SMILES: COC(=O)[C@]12Cc3cnn(-c4ccc(F)cc4)c3C=C1CCN(C(=O)OC(C)(C)C)C2]

and
   2-bromo-pyridine:

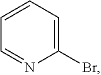

wherein the pyridine is present in a molar ratio of about 3.0 to the compound of Formula III, and wherein the Grignard reagent is present in a molar ratio of about 3.0 to the compound of Formula III;
(c1) adding acetic acid and water to the third reaction mixture to form a workup mixture;
(c2) distilling the workup mixture to form an intermediate mixture comprising a compound of Formula IIb:

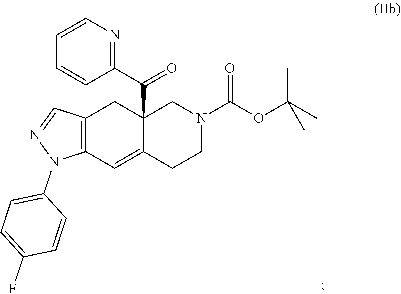 [SMILES: CC(C)(C)OC(=O)N1CCC2=Cc3c(cnn3-c3ccc(F)cc3)C[C@]2(C(=O)c2ccccn2)C1]

(b) forming a second reaction mixture comprising the intermediate mixture, acetonitrile, and methanesulfonic acid, to form a compound of Formula IIa having the structure:

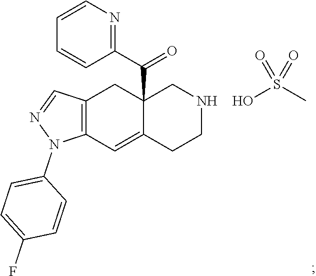

(a) forming a first reaction mixture comprising the compound of Formula IIa, triethylamine, ethyl acetate, and 4-(trifluoromethyl)benzenesulfonyl chloride:

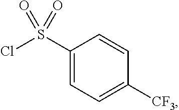 [SMILES: O=S(=O)(Cl)c1ccc(C(F)(F)F)cc1]

wherein the sulfonyl chloride is present in a molar ratio of about 1.0 to the compound of Formula IIa;
(a1) adding N-methylpiperazine to the first reaction mixture to remove unreacted 4-(trifluoromethyl)benzenesulfonyl chloride;
(a2) adding HCl and water to the first reaction mixture to partition the first reaction mixture into a first water mixture and a first organic mixture;
(a3) separating the first water mixture from the first organic mixture;
(a4) concentrating the first organic mixture;
(a5) adding ethanol to the concentrated first organic mixture; and
(a6) adding water to the concentrated first organic mixture to precipitate the compound of Formula I in a yield of at least 75% and a purity of at least 98%.

### IV. Method of Preparing Formula IIb from Formula III

The compound of Formula IIb can be prepared by any suitable method, such as the method described for Intermediate 11 of U.S. Pat. No. 8,859,774. In some embodiments, the present invention provides a method of preparing a compound of Formula IIb:

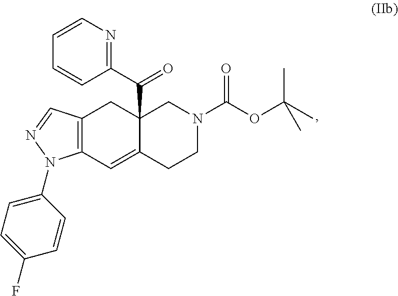 [SMILES: CC(C)(C)OC(=O)N1CCC2=Cc3c(cnn3-c3ccc(F)cc3)C[C@]2(C(=O)c2ccccn2)C1]

comprising the step of:
(c) forming a third reaction mixture comprising a Grignard reagent, a compound of Formula III:

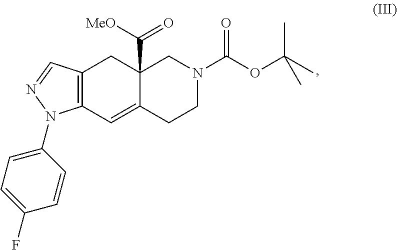 [SMILES: COC(=O)[C@]12Cc3cnn(-c4ccc(F)cc4)c3C=C1CCN(C(=O)OC(C)(C)C)C2]

and
   2-bromo-pyridine:

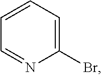

wherein the pyridine is present in a molar ratio of 2.8 to 3.2 to the compound of Formula III, and wherein the Grignard reagent is present in a molar ratio of 2.8 to 3.3 to the compound of Formula III, to prepare the compound of Formula IIb.

The Grignard reagent can be any suitable Grignard reagent. In some embodiments, the Grignard reagent comprises iPrMgCl or iPrMgBr. In some embodiments, the Grignard reagent comprises iPrMgBr. In some embodiments, the Grignard reagent comprises iPrMgCl.

The Grignard reagent can be present in any suitable molar ratio to the compound of Formula III. For example, the Grignard reagent can be present in a molar ratio of from 2.0 to 4.0, or 2.5 to 3.5, from 2.6 to 3.4, from 2.7 to 3.3, 2.8 to 3.3, 2.8 to 3.2, from 2.9 to 3.2, from 2.9 to 3.1 to the compound of Formula III. In some embodiments, the Grignard reagent can be present in a molar ratio of from 2.8 to 3.3 to the compound of Formula III. In some embodiments, the Grignard reagent can be present in a molar ratio of from 2.9 to 3.2 to the compound of Formula III. The Grignard reagent can be present in a molar ratio of about 2.90, or about 2.95, 3.00, 3.05, 3.10, 3.15, or about 3.20 to the compound of Formula III. In some embodiments, the Grignard reagent can be present in a molar ratio of about 3.0 to the compound of Formula III.

The pyridine can be present in any suitable ratio to the compound of Formula III. For example, the pyridine can be present in a molar ratio of 2.0 to 4.0, or 2.5 to 3.5, from 2.6 to 3.4, from 2.7 to 3.3, 2.8 to 3.2, from 2.9 to 3.1 to the compound of Formula III. In some embodiments, the pyridine can be present in a molar ratio of from 2.8 to 3.2 to the compound of Formula III. The pyridine can be present in a molar ratio of about 2.5, or about 2.6, 2.7, 2.8, 2.9, 3.0, 3.1, 3.2, 3.3, 3.4 or about 3.5 to the compound of Formula III. In some embodiments, the pyridine can be present in a molar ratio of about 3.0 to the compound of Formula III.

The third reaction mixture can also include a third solvent. The third solvent can be any suitable solvent including, but not limited to, tetrahydrofuran, 2-methyltetrahydrofuran, toluene xylene, or combinations thereof. In some embodiments, the third reaction mixture further comprises a third solvent. In some embodiments, the third solvent can be tetrahydrofuran, 2-methyltetrahydrofuran, toluene, xylene, or combinations thereof. In some embodiments, the third reaction mixture further comprises tetrahydrofuran and toluene.

In some embodiments, the method of preparing the compound of Formula IIb also includes: (c1) adding an acid and water to the third reaction mixture to form a workup mixture; and (c2) distilling the workup mixture to form an intermediate mixture comprising the compound of Formula IIb.

The acid of step (c1) can be any suitable acid. In some embodiments, the acid comprises formic acid, acetic acid, propanoic acid, butyric acid, hexanoic acid, octanoic acid, trifluoroacetic acid, or mixtures thereof. In some embodiments, the acid comprises acetic acid.

In some embodiments, the method of preparing the compound of Formula IIb comprises:
(c) forming the third reaction mixture comprising iPrMgCl, tetrahydrofuran, toluene, the compound of Formula III:

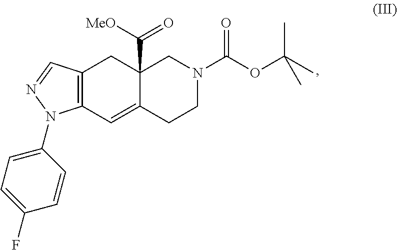 [SMILES: COC(=O)[C@]12Cc3cnn(-c4ccc(F)cc4)c3C=C1CCN(C(=O)OC(C)(C)C)C2]

and
   2-bromo-pyridine:

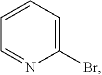

wherein the pyridine is present in the molar ratio of about 3.0 to the compound of Formula III, and wherein the Grignard reagent is present in the molar ratio of about 3.0 to the compound of Formula III, to prepare the compound of Formula IIb:

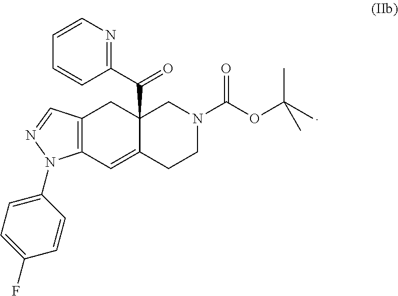 [SMILES: CC(C)(C)OC(=O)N1CCC2=Cc3c(cnn3-c3ccc(F)cc3)C[C@]2(C(=O)c2ccccn2)C1]

(c1) adding acetic acid and water to the third reaction mixture to form the workup mixture; and
(c2) distilling the workup mixture to form an intermediate mixture comprising the compound of Formula IIb.

### V. Low Impurity Compositions

The present invention provides compositions of Formula I having a low impurity content. The impurity content can be expressed in a variety of different methods. For example, the impurity content can be expressed as a % (HPLC peak area). In some embodiments, the impurity content can be expressed as a % (HPLC peak area). In some embodiments, the present invention provides a composition comprising:
a compound of Formula I in an amount of at least 99%:

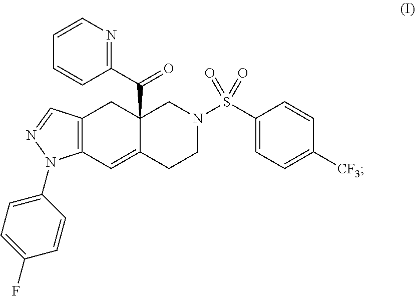 [SMILES: O=C(c1ccccn1)[C@]12Cc3cnn(-c4ccc(F)cc4)c3C=C1CCN(S(=O)(=O)c1ccc(C(F)(F)F)cc1)C2]

and
one or more impurity in an amount of from 0.01 to 1%.

The composition of Formula I can include one or more impurities present in a total amount of 0.01 to 1%. In some embodiments, the impurity includes at least one of:
a compound of Formula X-A in an amount of less than 0.5%:

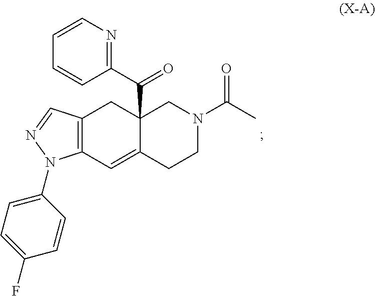 [SMILES: CC(=O)N1CCC2=Cc3c(cnn3-c3ccc(F)cc3)C[C@]2(C(=O)c2ccccn2)C1]

a compound of Formula X-C in an amount of less than 0.5%:

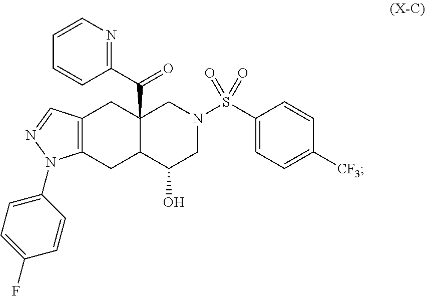 [SMILES: O=C(c1ccccn1)[C@]12Cc3cnn(-c4ccc(F)cc4)c3CC1[C@@H](O)CN(S(=O)(=O)c1ccc(C(F)(F)F)cc1)C2]

a compound of Formula X-5 in an amount of less than 0.1%:

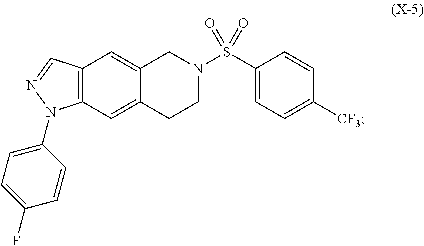 [SMILES: O=S(=O)(c1ccc(C(F)(F)F)cc1)N1CCc2cc3c(cnn3-c3ccc(F)cc3)cc2C1]

and
a compound of Formula X-6 in an amount of less than 0.1%:

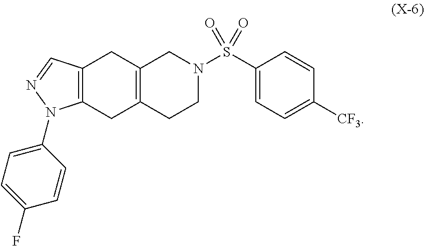 [SMILES: O=S(=O)(c1ccc(C(F)(F)F)cc1)N1CCC2=C(Cc3cnn(-c4ccc(F)cc4)c3C2)C1]

The impurity present in the composition of the compound Formula I can include the compound of Formula X-A in an amount of less than 1%. For example, the composition of the compound Formula I can include less than 1.0%, or less than 0.9, 0.8, 0.75, 0.7, 0.6, 0.5, 0.4, 0.3, 0.25, 0.2, or less than 0.1% of the compound of Formula X-A. In some embodiments, the composition comprising the compound of Formula I can contain less than 0.5% of the compound of Formula X-A. In some embodiments, the composition comprising the compound of Formula I can contain less than 0.3% of the compound of Formula X-A. In some embodiments, the composition comprising the compound of Formula I can contain less than 0.1% of the compound of Formula X-A.

The impurity present in the composition of the compound Formula I can include the compound of Formula X-C in an amount of less than 1%. For example, the composition of the compound Formula I can include less than 1.0%, or less than 0.9, 0.8, 0.75, 0.7, 0.6, 0.5, 0.4, 0.3, 0.25, 0.2, or less than 0.1% of the compound of Formula X-C. In some embodiments, the composition comprising the compound of Formula I can contain less than 0.5% of the compound of Formula X-C. In some embodiments, the composition comprising the compound of Formula I can contain less than 0.3% of the compound of Formula X-C. In some embodiments, the composition comprising the compound of Formula I can contain less than 0.2% of the compound of Formula X-C. In some embodiments, the composition comprising the compound of Formula I can contain less than 0.1% of the compound of Formula X-C. In some embodiments, the composition comprising the compound of Formula I can contain less than 0.05% of the compound of Formula X-C.

The impurity present in the composition comprising the compound of Formula I can contain less than 1.0%, or less than 0.9, 0.8, 0.75, 0.7, 0.6, 0.5, 0.4, 0.3, 0.25, 0.2, or less than 0.1% of the compound of Formula X-5. In some embodiments, the composition comprising the compound of Formula I can contain less than 0.25% of the compound of Formula X-5. In some embodiments, the composition comprising the compound of Formula I can contain less than 0.2% of the compound of Formula X-5. In some embodiments, the composition comprising the compound of Formula I can contain less than 0.1% of the compound of Formula X-5.

The impurity present in the composition comprising the compound of Formula I can contain less than 1.0%, or less than 0.9, 0.8, 0.75, 0.7, 0.6, 0.5, 0.4, 0.3, 0.25, 0.2, or less than 0.1% of the compound of Formula X-6. In some embodiments, the composition comprising the compound of Formula I can contain less than 0.25% of the compound of Formula X-6. In some embodiments, the composition comprising the compound of Formula I can contain less than 0.2% of the compound of Formula X-6. In some embodiments, the composition comprising the compound of Formula I can contain less than 0.1% of the compound of Formula X-6.

In some embodiments, the impurity comprises: the compound of Formula X-A in an amount of less than 0.1%; the compound of Formula X-C in an amount of less than 0.2%; the compound of Formula X-5 in an amount of less than 0.1%; and the compound of Formula X-6 in an amount of less than 0.1%.

The impurity present in the composition comprising the compound of Formula I can contain ethyl 4-(trifluoromethyl)benzene sulfonate in an amount of less than 100 ppm. For example, the composition comprising the compound of Formula I can contain the impurity of ethyl 4-(trifluoromethyl)benzene sulfonate in an amount of less than 100 ppm, or less than 90, 80, 75, 60, 50, 40, 30, or less than 25 ppm. In some embodiments, the composition comprising the compound of Formula I can contain the impurity of ethyl 4-(trifluoromethyl)benzene sulfonate in an amount of less than 25 ppm.

The impurity present in the composition comprising the compound of Formula I can contain 4-(trifluoromethyl)benzene sulfonyl chloride in an amount of less than 100 ppm. For example, the composition comprising compound of Formula I can contain 1-methyl-1H-pyrazole-4-sulfonyl chloride in an amount of less than 100 ppm, or less than 90, 80, 75, 60, 50, 40, 30, 25, 20, 15 or less than 10 ppm. In some embodiments, the composition comprising compound of Formula I can contain 4-(trifluoromethyl)benzene sulfonyl chloride in an amount of less than 10 ppm.

In some embodiments, the impurity further comprises: 4-(trifluoromethyl)benzene sulfonyl chloride in an amount of less than 10 ppm; and ethyl 4-(trifluoromethyl)benzene sulfonate in an amount of less than 25 ppm.

In some embodiments, the composition comprising the compound of Formula I can also contain a compound of Formula X-B in an amount of less than 0.1%:

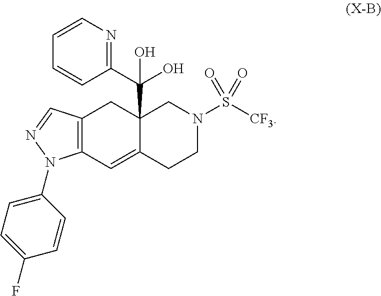 [SMILES: O=S(=O)(N1CCC2=Cc3c(cnn3-c3ccc(F)cc3)C[C@]2(C(O)(O)c2ccccn2)C1)C(F)(F)F]

In some embodiments, the composition comprising the compound of Formula I can also contain one or more of the following impurities:
a compound of Formula X-D in an amount of less than 0.2%:

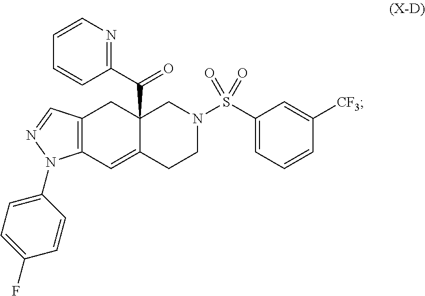 [SMILES: O=C(c1ccccn1)[C@]12Cc3cnn(-c4ccc(F)cc4)c3C=C1CCN(S(=O)(=O)c1cccc(C(F)(F)F)c1)C2]

and
a compound of Formula X-E in an amount of less than 0.1%:

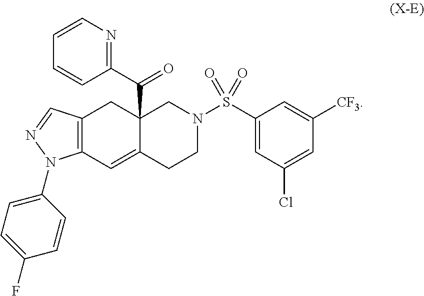 [SMILES: O=C(c1ccccn1)[C@]12Cc3cnn(-c4ccc(F)cc4)c3C=C1CCN(S(=O)(=O)c1cc(Cl)cc(C(F)(F)F)c1)C2]

In some embodiments, the composition comprising the compound of Formula I can also contain a compound of Formula X-H in an amount of less than 0.15%:

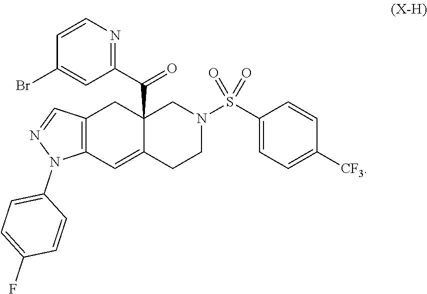 [SMILES: O=C(c1cc(Br)ccn1)[C@]12Cc3cnn(-c4ccc(F)cc4)c3C=C1CCN(S(=O)(=O)c1ccc(C(F)(F)F)cc1)C2]

In some embodiments, the composition comprising the compound of Formula I can also contain one or more of the following impurities:
a compound of Formula X-D in an amount of less than 0.2%:

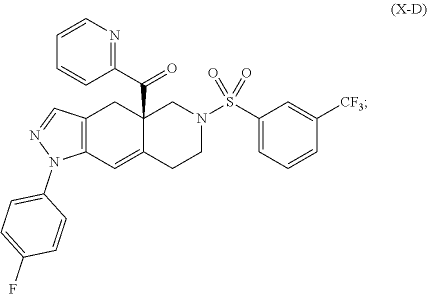 [SMILES: O=C(c1ccccn1)[C@]12Cc3cnn(-c4ccc(F)cc4)c3C=C1CCN(S(=O)(=O)c1cccc(C(F)(F)F)c1)C2]

a compound of Formula X-E in an amount of less than 0.1%:

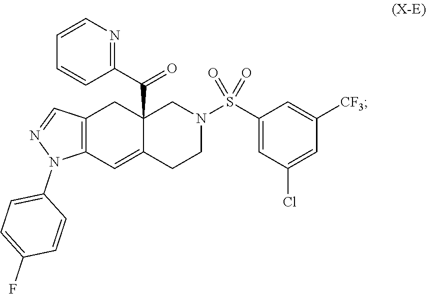 [SMILES: O=C(c1ccccn1)[C@]12Cc3cnn(-c4ccc(F)cc4)c3C=C1CCN(S(=O)(=O)c1cc(Cl)cc(C(F)(F)F)c1)C2]

a compound of Formula X-H in an amount of less than 0.15%:

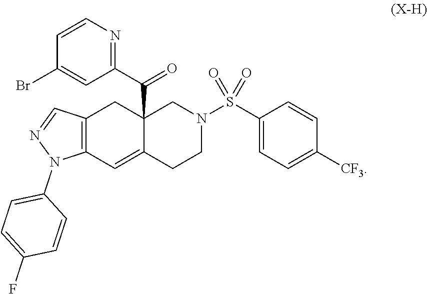 [SMILES: O=C(c1cc(Br)ccn1)[C@]12Cc3cnn(-c4ccc(F)cc4)c3C=C1CCN(S(=O)(=O)c1ccc(C(F)(F)F)cc1)C2]

### VI. Crystalline Forms of Formula IIa

The present invention also provides crystalline salt forms of the compound of Formula IIa, including, but not limited to, methanesulfonic acid, dibenzoyl-L-tartaric acid, 4-nitrobenzoic acid and (1S)-(+)-camphorsulfonic acid crystalline salts. In some embodiments, the present invention provides a crystalline form of (R)-(1-(4-fluorophenyl)-1,4,5,6,7,8-hexahydro-4aH-pyrazolo[3,4-g]isoquinolin-4a-yl)(pyridin-2-yl)methanone that is:
(R)-(1-(4-fluorophenyl)-1,4,5,6,7,8-hexahydro-4aH-pyrazolo[3,4-g]isoquinolin-4a-yl)(pyridin-2-yl)methanone methanesulfonic acid;
(R)-(1-(4-fluorophenyl)-1,4,5,6,7,8-hexahydro-4aH-pyrazolo[3,4-g]isoquinolin-4a-yl)(pyridin-2-yl)methanone camphorsulfonic acid;
(R)-(1-(4-fluorophenyl)-1,4,5,6,7,8-hexahydro-4aH-pyrazolo[3,4-g]isoquinolin-4a-yl)(pyridin-2-yl)methanone dibenzoyl-L-tartaric acid; or
(R)-(1-(4-fluorophenyl)-1,4,5,6,7,8-hexahydro-4aH-pyrazolo[3,4-g]isoquinolin-4a-yl)(pyridin-2-yl)methanone 4-nitrobenzoic acid.

In some embodiments, the present invention provides a crystalline form of (R)-(1-(4-fluorophenyl)-1,4,5,6,7,8-hexahydro-4aH-pyrazolo[3,4-g]isoquinolin-4a-yl)(pyridin-2-yl)methanone methanesulfonic acid:

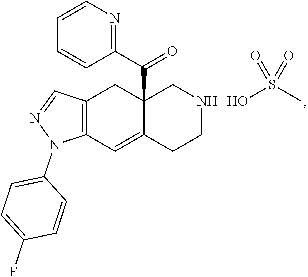

characterized by an X-ray powder diffraction (XRPD pattern having peaks at about 7.8, 14.7, and 15.5° 2-θ±0.2° 2-θ.

In some embodiments, the XRPD further comprises peaks at about 11.6, 21.3, 22.4, and 23.2° 2-θ±0.2° 2-θ. In some embodiments, the XRPD further comprises peaks at about 12.1, 14.4, 17.1, 20.4, 20.7, 20.9, 22.4, 26.2, 26.8, and 27.6° 2-θ±0.2° 2-θ. In some embodiments, the XRPD further comprises peaks at about 7.8, 11.6, 12.1, 14.4, 14.7, 15.5, 17.1, 20.4, 20.7, 20.9, 21.3, 22.4, 23.2, 26.2, 26.8, and 27.6° 2-θ±0.2° 2 θ. In some embodiments, the crystalline form is characterized by the XRPD pattern substantially as shown in Table 2. In some embodiments, the crystalline form is characterized by the XRPD pattern substantially as shown in FIG. 2.

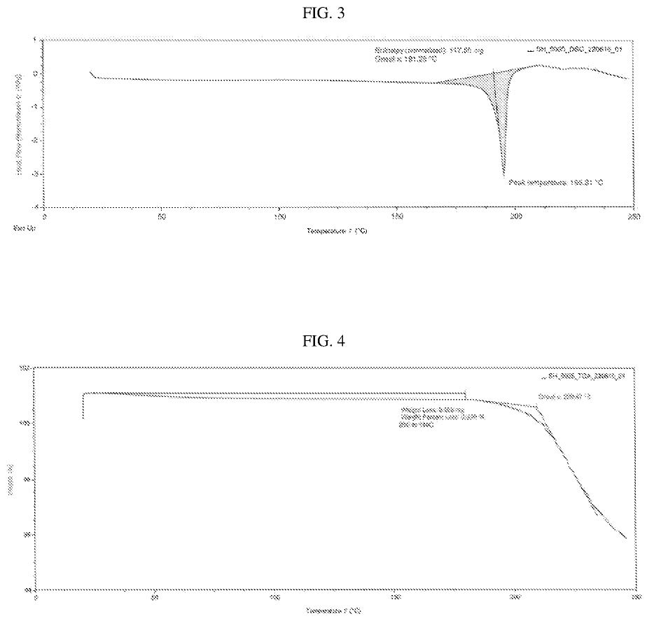

In some embodiments, the crystalline form is characterized by a differential scanning calorimetry (DSC) thermogram having an endotherm with an onset of about 191° C. In some embodiments, the crystalline form is characterized by a differential scanning calorimetry (DSC) thermogram substantially as shown in FIG. 3.

In some embodiments, the crystalline form is characterized by a thermal gravimetric analysis (TGA) substantially as shown in FIG. 4.

In some embodiments, the present invention provides a crystalline form of (R)-(1-(4-fluorophenyl)-1,4,5,6,7,8-hexahydro-4aH-pyrazolo[3,4-g]isoquinolin-4a-yl)(pyridin-2-yl)methanone camphorsulfonic acid. In some embodiments, the present invention provides a crystalline form of (R)-(1-(4-fluorophenyl)-1,4,5,6,7,8-hexahydro-4aH-pyrazolo[3,4-g]isoquinolin-4a-yl)(pyridin-2-yl)methanone dibenzoyl-L-tartaric acid. In some embodiments, the present invention provides a crystalline form of (R)-(1-(4-fluorophenyl)-1,4,5,6,7,8-hexahydro-4aH-pyrazolo[3,4-g]isoquinolin-4a-yl)(pyridin-2-yl)methanone 4-nitrobenzoic acid.

### VII. Compositions

In some embodiments, the present invention provides a pharmaceutical composition comprising a low impurity composition of the present invention and a pharmaceutically acceptable excipient.

The low impurity compositions of the present invention can be prepared and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. The low impurity compositions of the present invention can also be administered by injection, that is, intravenously, intramuscularly, intracutaneously, subcutaneously, intraduodenally, or intraperitoneally. Also, the compounds described herein can be administered by inhalation, for example, intranasally. Additionally, the low impurity compositions of the present invention can be administered transdermally. The compounds of formula I of this invention can also be administered by in intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present invention also provides pharmaceutical compositions including one or more pharmaceutically acceptable carriers and/or excipients and a compound of formula I.

For preparing pharmaceutical compositions from the low impurity compositions of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, surfactants, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties and additional excipients as required in suitable proportions and compacted in the shape and size desired.

The powders, capsules and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other exceipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers including, but not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain the compounds of formula I mixed with a filler or binders such as lactose or starches, lubricants such as talc or magnesium stearate, and, optionally, stabilizers. In soft capsules, the compounds of formula I may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of formula I in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The low impurity compositions of the present invention can be delivered by transdermally, by a topical route, formulated as applicator sticks, solutions, suspensions, emulsions, gels, creams, ointments, pastes, jellies, paints, powders, and aerosols.

The low impurity compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In some embodiments, the formulations of the low impurity compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the GR modulator into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. For example, the dose can be 50 mg, or 100, 150, 200, 250, 300, 350, 400, 450, or 500 mg. The composition can, if desired, also contain other compatible therapeutic agents.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, GR and/or MR modulator and disease or condition treated.

Single or multiple administrations of the formulations can be administered depending on the dosage and frequency as required and tolerated by the patient. The formulations should provide a sufficient quantity of active agent to effectively treat the disease state. Thus, in one embodiment, the pharmaceutical formulations for oral administration of the low impurity compositions is in a daily amount of between about 0.5 to about 30 mg per kilogram of body weight per day. In an alternative embodiment, dosages are from about 1 mg to about 20 mg per kg of body weight per patient per day are used. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration. Actual methods for preparing formulations including the compound of formula I for parenteral administration are known or apparent to those skilled in the art and are described in more detail in such publications as Remington's, supra. See also Nieman, In “Receptor Mediated Antisteroid Action,” Agarwal, et al., eds., De Gruyter, New York (1987).

The low impurity compositions described herein can be used in combination with one another, with other active agents known to be useful in modulating a glucocorticoid receptor, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In some embodiments, the active agents can be formulated separately. In some embodiments, the active and/or adjunctive agents may be linked or conjugated to one another.

After a pharmaceutical composition including a compound of formula I of the invention has been formulated in one or more acceptable carriers, it can be placed in an appropriate container and labeled for treatment of an indicated condition. For administration of the compounds of formula I, such labeling would include, e.g., instructions concerning the amount, frequency and method of administration.

In some embodiments, the low impurity compositions of the present invention are useful for parenteral administration, such as intravenous (IV) administration or administration into a body cavity or lumen of an organ. The formulations for administration will commonly comprise a solution of the compositions of the present invention dissolved in one or more pharmaceutically acceptable carriers. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, tonicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

### VIII. Methods & Use

In some embodiments, the present invention provides a method of treating a disorder or condition through modulating a glucocorticoid receptor, the method comprising administering to a subject in need of such treatment, a therapeutically effective amount of any one of the low impurity compositions of the present invention, or a pharmaceutical composition of the present invention, thereby treating the disorder or condition.

In some embodiments, the present invention provides a method of treating a disorder or condition through antagonizing a glucocorticoid receptor, the method comprising administering to a subject in need of such treatment, an effective amount of any one of the low impurity compositions of the present invention, or a pharmaceutical composition of the present invention.

In some embodiments, the disorder or condition is selected from the group consisting of amyotrophic lateral sclerosis (ALS), obesity, diabetes, cardiovascular disease, hypertension, Syndrome X, depression, anxiety, glaucoma, neurodegeneration, Alzheimer's disease, Parkinson's disease, Cushing's Syndrome, Cushing Disease, cancer, liver disease, osteoporosis, muscle frailty, a disorder caused by adrenal disease-related cortisol excess, addiction, psychosis, anorexia, cachexia, post-traumatic stress syndrome, post-surgical bone fracture, a GR-related metabolic disorders, major psychotic depression, mild cognitive impairment, dementia, hyperglycemia, a stress disorder, antipsychotic induced weight gain, delirium, cognitive impairment in depressed patients, postpartum psychosis, postpartum depression, and a neurological disorder in a premature infant.

In some embodiments, the method includes administering one or more second agents (e.g. therapeutic agents). In some embodiments, the method includes administering one or more second agents (e.g. therapeutic agents) in a therapeutically effective amount. In some embodiments, the second agent is an agent known to be useful in modulating a glucocorticoid receptor. In some embodiments, the second agent is an agent for treating amyotrophic lateral sclerosis (ALS), obesity, diabetes, cardiovascular disease, hypertension, Syndrome X, depression, anxiety, glaucoma, neurodegeneration, Alzheimer's disease, Parkinson's disease, Cushing's Syndrome, Cushing Disease, cancer, liver disease, osteoporosis, muscle frailty, a disorder caused by adrenal disease-related cortisol excess, addiction, psychosis, anorexia, cachexia, post-traumatic stress syndrome, post-surgical bone fracture, a GR-related metabolic disorders, major psychotic depression, mild cognitive impairment, dementia, hyperglycemia, a stress disorder, antipsychotic induced weight gain, delirium, cognitive impairment in depressed patients, postpartum psychosis, postpartum depression, and a neurological disorder in a premature infant. In some embodiments, the second agent is an agent for treating major psychotic depression, stress disorders or antipsychotic induced weight gain. In some embodiments, the second agent is an agent for treating nonalcoholic fatty liver disease and/or nonalcoholic steatohepatitis. In some embodiments, the second agent is an agent for treating an addiction disorder. In some embodiments, the second agent is an agent for treating cancer. In some embodiments, the second agent is an anti-cancer agent. In some embodiments, the second agent is a chemotherapeutic.

In some embodiments, any one of the low impurity compositions of the present invention, or a pharmaceutical composition of the present invention can be used for a method of treating a disorder or condition through modulating a glucocorticoid receptor.

In some embodiments, any one of the low impurity compositions of the present invention, or a pharmaceutical composition of the present invention can be used for a method of treating a disorder or condition through antagonizing a glucocorticoid receptor.

In some embodiments, any one of the low impurity compositions of the present invention, or a pharmaceutical composition of the present invention, can be used in the manufacture of a medicament for treating a disorder or condition through modulating a glucocorticoid receptor.

In some embodiments, any one of the low impurity compositions of the present invention, or a pharmaceutical composition of the present invention, can be used in the manufacture of a medicament for treating a disorder or condition through antagonizing a glucocorticoid receptor.

In some embodiments, the present invention provides a method of treating amyotrophic lateral sclerosis (ALS), comprising administering to a subject in need thereof, a therapeutically effective amount of a low impurity composition of the present invention, or a pharmaceutical composition of the present invention, thereby treating fatty liver disease.

In some embodiments, any one of the low impurity compositions of the present invention, or a pharmaceutical composition of the present invention, can be used in the manufacture of a medicament for treating amyotrophic lateral sclerosis (ALS).

### IX. Examples

The following abbreviations are used in the methods below:

| | |
|---|---|
| ° C. | Degree Celsius |
| ACN, MeCN | acetonitrile |
| aq | Aqueous |
| atm | Atmospheric pressure |
| Barg | Bar(g); Pressure in bar as determined by guage |
| Boc | t-butyloxycarbonyl |
| DIPEA | N,N-diisopropylethylamine (iPr₂NEt) |
| EtOAc | Ethyl Acetate |
| eq or equiv | Equivalent |
| FA | Formic acid |
| g | Gram |
| HDPE | High density polyethylene |
| Kg | kilogram |
| L | Liter |
| M | Molar |
| mbar | millibar |
| mins | minutes |
| mL | milliliter |
| MSA | Methanesulfonic acid |
| MTBE | methyl t-butyl ether |
| nm | nanometers |
| NMR | Nuclear Magnetic Resonance |
| Parts | |
| PMI | Potential Mutagenic Impurity |
| ppm | Parts per million |
| r.t. | room temperature |
| THF | tetrahydrofuran |
| μm | micrometer |

X-ray Powder Diffraction (XRPD). XRPD analyses were performed using a Panalytical Xpert Pro diffractometer equipped with a Cu X-ray tube and a Pixcel detector system. The isothermal samples were analysed in transmission mode and held between low density polyethylene films. The XRPD program used included the following parameters: (1) range 3-40° 20, (2) step size 0.013°, (3) counting time 99 sec, and (4) about 22 min run time.

XRPD patterns were sorted using HighScore Plus 2.2c software.

Differential Scanning Calorimetry (DSC). DSC analyses were carried out on a Perkin Elmer Jade Differential Scanning Calorimeter. Accurately weighed samples were placed in crimped aluminium pans. Each sample was heated under nitrogen at a rate of 10° C./minute to a maximum of 300° C. Indium metal was used as the calibration standard. Temperatures were reported at the transition onset to the nearest 0.01 degree.

The reaction steps of the present invention can be performed for any suitable reaction time. For example, the reaction time can be for minutes, hours, or days. In some embodiments, the reaction time can be for several hours, such as at least eight hours. In some embodiments, the reaction time can be for several hours, such as at least overnight. In some embodiments, the reaction time can be for several days. In some embodiments, the reaction time can be for at least two hours. In some embodiments, the reaction time can be for at least eight hours. In some embodiments, the reaction time can be for at least several days. In some embodiments, the reaction time can be for about two hours, or for about 4 hours, or for about 6 hours, or for about 8 hours, or for about 10 hours, or for about 12 hours, or for about 14 hours, or for about 16 hours, or for about 18 hours, or for about 20 hours, or for about 22 hours, or for about 24 hours. In some embodiments, the reaction time can be for about 1 day, or for about two days, or for about three days, or for about four days, or for about five days, or for about six days, or for about a week, or for about more than a week.

The reaction steps of the present invention can be performed at any suitable reaction temperature. Representative temperatures include, but are not limited to, below room temperature, at room temperature, or above room temperature. Other temperatures useful in the methods of the present invention include from about −40° C. to about 65° C., or from about room temperature to about 40° C., or from about 40° C. to about 65° C., or from about 40° C. to about 60° C. In some embodiments, the reaction mixture can be at a temperature of about room temperature, or at a temperature of about 15° C., or at about 20° C., or at about 25° C. or at about 30° C., or at about 35° C., or at about 40° C., or at about 45° C., or at about 50° C., or at about 55° C., or at about 60° C., or at about 65° C.

### Example 1. Preparation of (R)-(1-(4-fluorophenyl)-6-((4-(trifluoromethyl)phenyl)sulfonyl)-4,4a,5,6,7,8-hexahydro-1H-pyrazolo[3,4-g]isoquinolin-4a-yl)(pyridin-2-yl)methanone

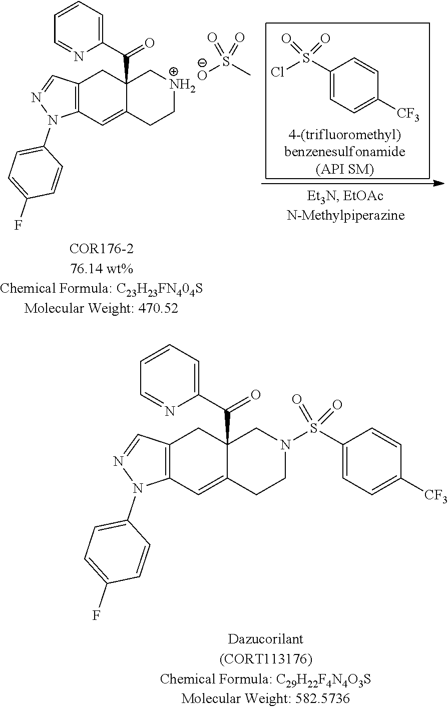

Two 400 L vessels were boiled out with ethyl acetate prior to use (A and B). Vessel A was used for the reaction and vessel B was fitted with an inline filter and used for precipitation of dazucorilant (CORT113176). COR176-2 [15.692 kg, (11.948 kg, 31.91 moles corrected)] and ethyl acetate [213.8 kg, 235 L, 15 vol] were charged to vessel A. The contents were agitated for 5 minutes then cooled to 0° C.

Triethylamine anhydrous [6.81 kg, 67.30 moles, 2.1 eq] was charged to vessel A over 17 minutes, maintaining a batch temperature below 10° C. The contents of vessel A were aged for 51 minutes at 0° C.

4-(Trifluoromethyl)benzenesulfonyl chloride [7.960 kg, 32.54 moles, 1 eq.] in ethyl acetate [27.4 kg, 31.4 L, 2 vol.] was charged to vessel A over 40 minutes, maintaining a batch temperature below 10° C. The batch was aged for 2 hours at 0° C. then analysed for conversion of COR176-2 to dazucorilant.

N-Methylpiperazine [0.808 kg, 8.07 moles, 25 mol %] was charged to vessel A. The batch was warmed to 20° C. and aged for 3 hours.

Hydrochloric acid solution [0.5 M; 79 kg, 5 vols.] was charged to vessel A at 20° C., and contents aged for 10 minutes. The aqueous layer was discarded and the organic layer was washed again with hydrochloric acid solution [0.5 M; 79 kg, 5 vols.], the contents aged for at least 10 minutes and the aqueous layer was discarded.

Purified water [79 kg, 5 vols.] was charged to the vessel, the organics were washed for at least 10 minutes, the biphasic mixture was allowed to settle, and the aqueous layer drained. This step was repeated twice.

The solution of dazucorilant was discharged into drums and analysed by HPLC for assay yield (for monitoring purposes only). No 1-methyl-4-((4-(trifluoromethyl)phenyl)sulfonyl)piperazine observed in assay yield HPLC traces (<0.04 A %).

An extraneous matter test was performed on vessel B with the oyster filter and API hoses as part of the train using the ethyl acetate from the initial boil-out. This was to ensure that no solid contamination of the final API occurred.

The dazucorilant solution was transferred to vessel B via a 1 micron in-line filter cartridge. The organic stream was concentrated from approximately 260 L to 31 L [2 vol] under reduced pressure, maintaining the batch temperature below 45° C. Ethanol [150 kg, 12 vol] was charged to vessel B via a 1 micron in-line filter cartridge. The solution was then concentrated from approximately 220 L to 47 L [3 vol] under reduced pressure, maintaining the batch temperature below 45° C. The dazucorilant solution was cooled to 20° C. then analysed for ethyl acetate content by GC.

The dazucorilant solution was transferred into a clean plastic lined drum [50 kg]. Ethanol [15.7 kg, 1.25 vols.] was charged to vessel B via a 1 micron in-line filter cartridge, the contents agitated for 5 minutes and combined with the dazucorilant solution. Ethanol [50 kg, 3 vols.] was charged to vessel B via a 1 micron in-line filter cartridge, the contents agitated and then discarded into a drum for disposal. Purified water [236 kg, 15 vols.] was charged to vessel B via a 1 micron in-line filter cartridge. The dazucorilant solution was charged to vessel B over 38 minutes with an agitation speed of 170 rpm. The contents of vessel B were aged for 12 hours then the liquors analysed for liquor loss (for information only).

The slurry was filtered, washing the filter cake with purified water [47 kg, 3 vol]. The solid was dried under a stream of nitrogen for 5 hours, and then in a vacuum oven at 60° C. for 64 hours. 18.46 kg (99.3% with respect to COR176-2) of dazucorilant as a white solid.

TABLE 1
Comparison of analytical data for batches measured on API release method

| | | | Example 1, | Example 1, |
|---|---|---|---|---|
| | Identity | Formula | Batch 1 | Batch 2 |
| LCAP Data | RRT 0.41 | X-A | <0.05 | ND |
| (A %) | RRT 0.49 | X-B | 0.08 | 0.09 |
| | RRT 0.61 | ND | ND | ND |
| | RRT 0.75 | X-C | 0.14 | 0.05 |
| | COR176-6 (Dihydro) | X-6 | ND | ND |
| | RRT 0.98 | X-D | 0.11 | 0.11 |
| | dazucorilant | I | 99.6 | 99.4 |
| | CORT125863 | X-5 | <0.05 | 0.06 |
| | RRT 1.13 | X-E | 0.06 | 0.07 |
| | RRT 1.15 | X-H | | 0.14 |
| PMI Data | 4-(trifluoromethyl) | | <6 ppm | <LOQ (18 ppm) |
| (ppm) | benzenesulfonyl | | | |
| | chloride | | | |
| | Ethyl 4- | | <18 ppm | <LOD (6 ppm) |
| | (trifluoromethyl) | | | |
| | benzenesulfonate | | | |

The characterization data of the title compound was consistent with that of Example 1 of U.S. Pat. No. 8,859,774.

### Example 2. Preparation of (R)-1-(4-fluorophenyl)-4a-picolinoyl-4,4a,5,6,7,8-hexahydro-1H-pyrazolo[3,4-g]isoquinolin-6-ium methanesulfonate

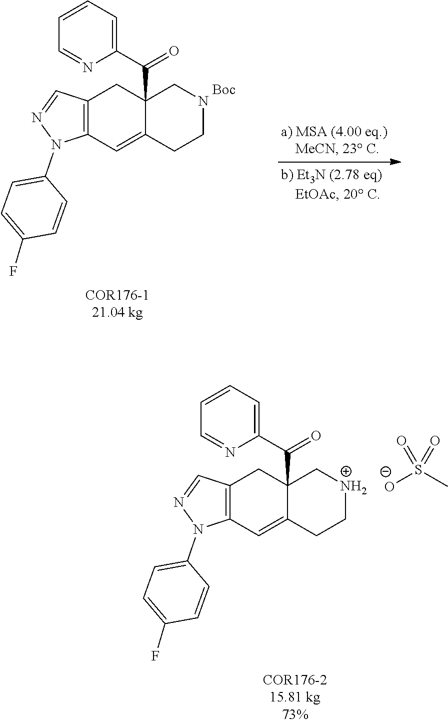

### Method A

A 400 L vessel was rinsed with toluene prior to use. COR176-1 [21.04 kg, 44.3 mol, 1.0 eq] as a stream in toluene [259.8 kg] was concentrated to 42 L [2 vol] under reduced pressure, maintaining a batch temperature below 45° C.

MeCN [166.1 kg] was added and the batch was concentrated from approximately 200 L to 42 L [2 vol] under reduced pressure, maintaining a batch temperature below 45° C. MeCN [27.0 kg] was added and a sample was taken to measure the content of toluene by GC.

The batch was cooled to 9.7° C. and MSA [17.14 kg, 178.3 mol, 4.0 eq] was added over 1 hour maintaining an internal batch temperature below 15° C. The batch was aged for 16 hours at 23-24° C. after which a sample was taken to measure reaction conversion by HPLC.

The batch was cooled to 9° C. and Et₃N [9.47 kg, 93.6 mol, 2.1 eq.] was added over 25 minutes maintaining an internal temperature below 15° C.

The batch was seeded with a slurry of COR176-2 (102 g, 0.2 mol, 0.5 mol %) in MeCN [300 g] and aged at 16° C. for 30 minutes.

Et₃N [3.06 kg, 30.2 mol, 0.68 eq.] was added over 30 minutes maintaining an internal temperature below 20° C. The batch was aged for 1 hour at 23° C. and subsequently was added ethyl acetate [114 kg, 6 vols.] over 20 minutes. The batch was cooled to −19.7° C. over at least 2 hours after which a sample was taken to measure liquor loss by HPLC.

The batch was filtered, then washed twice with THF [46.8 kg, 2.5 vols. & 37.1 kg, 2 vols] and dried under a flow of nitrogen for 1 hour. The cake was dried under vacuum at 40° C. for 14 hours with a minimal nitrogen sweep and analysed for weight percent. 15.81 kg of isolated COR176-2 obtained (76.14 wt % COR176-2 (free base) in salt, corresponding to 73% isolated yield).

### Method B

A 3 L round bottom flask was charged with 2151.22 g of COR176-1 in toluene [8.9% COR176-1, 191.46 g COR176-1]. The mixture was concentrated to 380 mL [2 vol] under reduced pressure, maintaining a batch temperature below 45° C.

MeCN [1493 g, 1900 mL] was added and the mixture was concentrated to 380 mL [2 vol] under reduced pressure, maintaining a batch temperature below 45° C. MeCN [239 g, 304 mL, 1.6 vol] was added and a sample was taken to measure the content of toluene by GC. The mixture was transferred to a 2 L Radleys reactor with nitrogen purging, and cooled to 10° C.

Methanesulfonic acid [155 g, 105 mL, 4.0 eq.] was added over 1 hour maintaining an internal batch temperature below 15° C. The batch was aged for 14 hours at 22° C. after which a sample was taken to measure reaction conversion by HPLC.

The batch was cooled to 10° C. and Et₃N [85 g, 117 mL, 2.1 eq.] was added over at least 30 minutes maintaining an internal temperature below 15° C.

The batch was seeded with a slurry of COR176-2 (0.94 g, 0.5 mol %) and aged at 15° C. for 30 minutes.

Et₃N [28 g, 38 mL, 0.68 eq.] was added over 30 minutes maintaining an internal temperature below 20° C. The batch was aged for 1 hour at 20° C. and subsequently was added ethyl acetate [1028 g, 1140 mL, 6 vols.] over 20 minutes. The batch was cooled to −20° C. over at least 2 hours after which a sample was taken to measure liquor loss by HPLC.

The batch was filtered, then washed twice with THF [425 g, 475 mL, 2.5 vols. & 338 g, 380 mL, 2 vols] and dried under a flow of nitrogen for 1 hour. The cake was dried under vacuum at 40° C. for 12 hours with a minimal nitrogen sweep and analysed for weight percent. 149.12 g of isolated COR176-2 obtained (79% isolated yield).

TABLE 2
XRPD Peak Table for Crystal form of (R)-1-(4-fluorophenyl)-
4a-picolinoyl-4,4a,5,6,7,8-hexahydro-1H-pyrazolo[3,4-
g]isoquinolin-6-ium methanesulfonate

| | | Relative |
|---|---|---|
| Angle | d Value | Intensity |

| | | | |
|---|---|---|---|
| 6.040° | 14.62046 | A | 0.9% |
| 7.767° | 11.37340 | A | 60.5% |
| 9.426° | 9.37492 | A | 0.7% |
| 11.560° | 7.64886 | A | 15.0% |
| 12.104° | 7.30623 | A | 5.6% |
| 14.356° | 6.16467 | A | 7.6% |
| 14.660° | 6.03777 | A | 100.0% |
| 15.547° | 5.69490 | A | 34.8% |
| 16.466° | 5.37936 | A | 4.9% |
| 17.085° | 5.18564 | A | 9.3% |
| 18.188° | 4.87362 | A | 1.5% |
| 18.559° | 4.77702 | A | 2.6% |
| 18.760° | 4.72631 | A | 2.9% |
| 20.220° | 4.38816 | A | 4.6% |
| 20.408° | 4.34829 | A | 7.7% |
| 20.683° | 4.29107 | A | 9.8% |
| 20.922° | 4.24246 | A | 8.8% |
| 21.322° | 4.16388 | A | 13.0% |
| 21.738° | 4.08513 | A | 3.5% |
| 22.406° | 3.96470 | A | 12.4% |
| 23.218° | 3.82787 | A | 17.9% |
| 23.785° | 3.73790 | A | 2.8% |
| 24.287° | 3.66182 | A | 0.7% |
| 24.772° | 3.59123 | A | 0.6% |
| 25.337° | 3.51243 | A | 3.9% |
| 25.786° | 3.45230 | A | 1.3% |
| 26.216° | 3.39655 | A | 5.8% |
| 26.758° | 3.32898 | A | 5.2% |
| 27.600° | 3.22937 | A | 6.9% |
| 28.289° | 3.15216 | A | 3.3% |
| 28.926° | 3.08424 | A | 4.7% |
| 29.499° | 3.02557 | A | 3.0% |
| 30.368° | 2.94094 | A | 3.0% |
| 31.911° | 2.80221 | A | 2.1% |
| 32.968° | 2.71472 | A | 1.5% |
| 33.823° | 2.64801 | A | 1.1% |
| 34.674° | 2.58496 | A | 0.4% |
| 35.995° | 2.49309 | A | 0.7% |
| 36.360° | 2.46887 | A | 1.7% |
| 36.710° | 2.44615 | A | 3.2% |
| 37.405° | 2.40226 | A | 1.9% |
| 39.363° | 2.28717 | A | 2.2% |

### Example 3. Preparation of (R)-1-(4-fluorophenyl)-4a-picolinoyl-4,4a,5,6,7,8-hexahydro-1H-pyrazolo[3,4-g]isoquinolin-6-ium acid salts

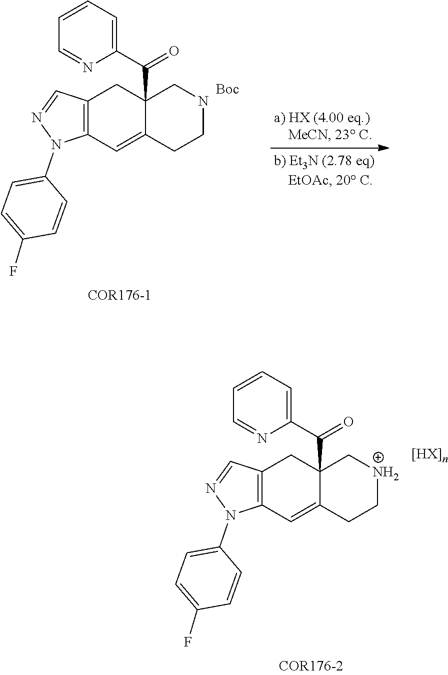

Additional crystalline acid salts of (R)-1-(4-fluorophenyl)-4a-picolinoyl-4,4a,5,6,7,8-hexahydro-1H-pyrazolo[3,4-g]isoquinolin-6-ium were prepared using dibenzoyl-L-tartaric acid, 4-nitrobenzoic acid, and (1S)-(+)-camphorsulfonic acid.

### Example 4. Preparation of tert-butyl (R)-1-(4-fluorophenyl)-4a-picolinoyl-1,4,4a,5,7,8-hexahydro-6H-pyrazolo[3,4-g]isoquinoline-6-carboxylate

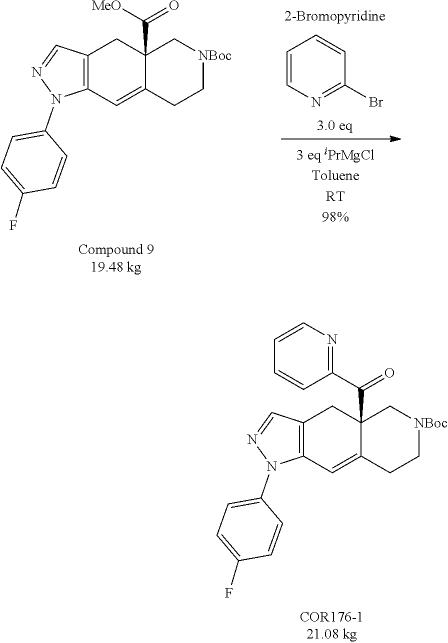 [SMILES: COC(=O)[C@]12Cc3cnn(-c4ccc(F)cc4)c3C=C1CCN(C(=O)OC(C)(C)C)C2 ; Brc1ccccn1 ; CC(C)(C)OC(=O)N1CCC2=Cc3c(cnn3-c3ccc(F)cc3)C[C@]2(C(=O)c2ccccn2)C1]

A 1000 L vessel was boiled out with toluene prior to use. COMPOUND 9 can be prepared according to Example 33 of U.S. Pat. No. 7,928,237. COMPOUND 9 [19.48 kg, 45.6 mol, 1.0 eq] and toluene [84.6 kg, 97 L, 5 vols.] were charged to the vessel. The solid was then dissolved over 5 min at 19° C. and the resulting solution degassed three times under positive pressure of nitrogen (vessel pressurized to 1000 Barg and allowed to vent to atmospheric pressure). The solution of COMPOUND 9 was transferred into a clean plastic lined drum. The vessel was rinsed with toluene [67.6 kg, 78 L, 4 vols.] which was subsequently combined with the COMPOUND 9 solution.

2-bromopyridine [21.84 kg, 138.2 mol, 3.0 eq] and toluene [168.8 kg, 195 L, 10 vols.] were charged to the vessel. The 2-bromopyridine solution was degassed 3 times under positive pressure of nitrogen (vessel pressurized to 1000 Barg and allowed to vent to atmospheric pressure).

Isopropylmagnesium chloride ca. 20% in THF [68.4 kg, 137.0 mol, 3.0 eq] was charged to the vessel over 75 minutes, maintaining the batch temperature below 22° C. The resulting mixture was aged for at 16.5 hours at 24° C.

The solution of COMPOUND 9 was charged to the vessel over 37 minutes, maintaining the batch temperature below 22° C. The batch was aged for 1 hour then analysed for conversion of COMPOUND 9 by HPLC (for monitoring purposes only). Reaction aged for a total of 2 hours at 22° C.

The batch was cooled to 12° C. and charged with Acetic Acid [27.69 kg, 461.1 mol, 10.0 eq] over 1 hour, maintaining a batch temperature below 22° C. The quenched mixture was aged for 17.75 hours then a sample taken to monitor reaction profile by HPLC (for monitoring purposes only).

1M Hydrochloric acid solution [199 kg, 195 L, 10 vols.] was charged to the vessel, aged for 15 minutes, the biphasic mixture allowed to settle, and the aqueous layer drained. This step was repeated. Purified water [199 kg, 195 L, 10 vols.] was charged to the vessel, aged for 15 minutes, the biphasic mixture allowed to settle, and the aqueous layer drained. This step was repeated once.

The organic stream was concentrated from approximately 420 L to 293 L [15 vol] under reduced pressure, maintaining a batch temperature below 45° C. The organic stream was cooled to 20° C. and transferred into clean plastic lined drums. A sample was taken and analyzed for weight percentage of solution (8.1 wt % calculated—21.34 kg (98.7% assay yield) in 263.4 kg crude solution).

The characterization data of the title compound was consistent with that of Intermediate 11 of U.S. Pat. No. 8,859,774.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of preparing a compound of Formula I: 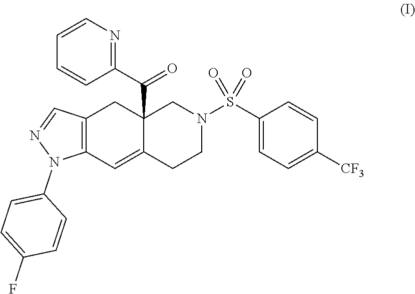 [SMILES: O=C(c1ccccn1)[C@]12Cc3cnn(-c4ccc(F)cc4)c3C=C1CCN(S(=O)(=O)c1ccc(C(F)(F)F)cc1)C2]
or a pharmaceutically acceptable salt thereof, comprising:
(a) forming a first reaction mixture comprising a compound of Formula II: 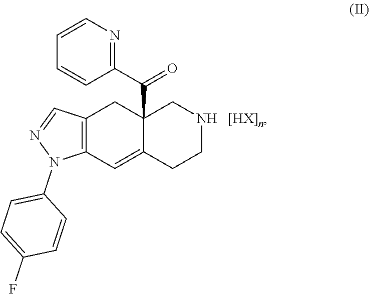 [SMILES: O=C(c1ccccn1)[C@@]12CNCCC1=Cc1c(cnn1-c1ccc(F)cc1)C2]
4-(trifluoromethyl)benzenesulfonyl chloride: 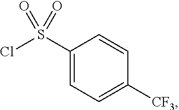 [SMILES: O=S(=O)(Cl)c1ccc(C(F)(F)F)cc1]
to prepare the compound of Formula I in a yield of at least 60% and a purity of at least 98%,
wherein
HX is an acid solvate; and
subscript n is 1 to 4.

2. The method of claim 1, wherein
HX is 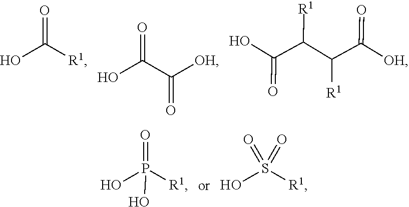
wherein
R¹ is C₁₋₆ alkyl, C₁₋₁₀ haloalkyl, phenyl, 4-methylphenyl, 4-NO₂-phenyl, —OC(O)— phenyl or

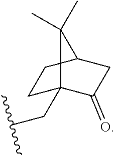 [SMILES: *CC12CCC(CC1=O)C2(C)C]

3.-4. (canceled)

5. The method of claim 1, wherein HX is MeS(O)₂OH.

6. The method of claim 1, comprising:
(a) forming the first reaction mixture comprising the compound of Formula IIa: 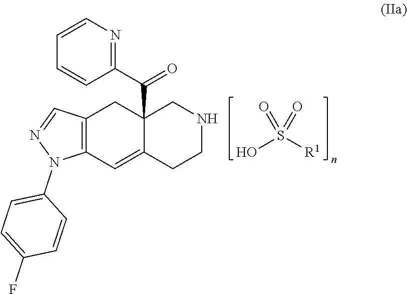
and
4-(trifluoromethyl)benzenesulfonyl chloride: 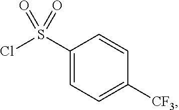 [SMILES: O=S(=O)(Cl)c1ccc(C(F)(F)F)cc1]
to prepare the compound of Formula I in a yield of at least 60% and a purity of at least 98%,
wherein
R¹ is C₁₋₆ alkyl, C₁₋₁₀ haloalkyl, phenyl, or 4-methylphenyl; and
subscript n is 1 to 4.

7. The method of claim 6, wherein the compound of Formula I is prepared in a yield of at least 75% and a purity of at least 98%.

8. The method of claim 6, wherein R¹ is C₁₋₂ alkyl, C₁₋₂ haloalkyl, phenyl, or 4-methylphenyl.

9. The method of claim 6, wherein R¹ is methyl, ethyl, —CF₃, phenyl, or 4-methylphenyl.

10. (canceled)

11. The method of claim 6, wherein n is 1.

12. The method of claim 1, wherein the first reaction mixture further comprises a non-nucleophilic amine base.

13. The method of claim 12, wherein the non-nucleophilic amine base comprises trimethylamine, triethylamine, N,N-diisopropyl ethylamine (DIPEA), N,N-dimethyl isopropylamine (DIMPA), 1-ethylpiperidine, N-methylmorpholine, N-methylpyrrolidine, pyridine, N,N-dimethylaniline, N,N-diethylaniline, 2,6-lutidine, 2,4,6-collidine, 4-dimethyl aminopyridine (DMAP), quinuclidine, 4-pyrrolidinopyridine, 1,4-diazabicyclo[2.2.2]octane (DABCO), or mixtures thereof.

14. (canceled)

15. The method of claim 1, wherein the first reaction mixture further comprises a first solvent.

16. The method of claim 15, wherein the first solvent comprises ethyl acetate, isopropyl acetate, or n-butyl acetate, or mixtures thereof.

17. (canceled)

18. The method of claim 6, wherein the sulfonyl chloride is present in a molar ratio of about 1.0 to the compound of Formula IIa.

19. The method of claim 1, further comprising the step of:
(a1) adding an amino scavenging agent to the first reaction mixture to remove unreacted 4-(trifluoromethyl)benzenesulfonyl chloride.

20. The method of claim 19, wherein the amino scavenging agent comprises N-methylpiperazine, N¹,N¹-dimethylethane-1,2-diamine, N¹,N¹,N²-trimethylethane-1,2-diamine, or N¹,N¹-bis(2-aminoethyl)ethane-1,2-diamine.

21. (canceled)

22. The method of claim 19, wherein the amino scavenging agent is present in a molar ratio of about 0.25 to the compound of Formula IIa.

23. The method of claim 19, further comprising the steps of:
(a2) adding a first acid and water to the first reaction mixture to partition the first reaction mixture into the first water mixture and the first organic mixture; and
(a3) separating the first water mixture from the first organic mixture.

24. The method of claim 23, wherein the first acid comprises hydrochloric acid.

25. The method of claim 23, further comprising the steps of:
(a4) concentrating the first organic mixture;
(a5) adding ethanol to the concentrated first organic mixture; and
(a6) adding water to the concentrated first organic mixture to precipitate the compound of Formula I.

26. The method of claim 1, comprising the steps of:
(a) forming the first reaction mixture comprising the compound of Formula IIa having the structure: 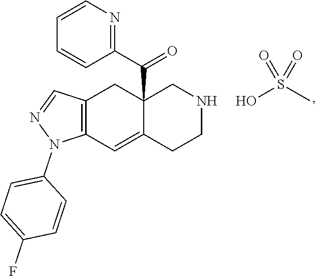
triethylamine, ethyl acetate, and 4-(trifluoromethyl)benzenesulfonyl chloride: 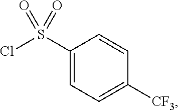 [SMILES: O=S(=O)(Cl)c1ccc(C(F)(F)F)cc1]
wherein the sulfonyl chloride is present in a molar ratio of about 1.0 to the compound of Formula IIa;
(a1) adding N-methylpiperazine to the first reaction mixture to remove unreacted 4-(trifluoromethyl)benzenesulfonyl chloride;
(a2) adding HCl and water to the first reaction mixture to partition the first reaction mixture into the first water mixture and the first organic mixture;
(a3) separating the first water mixture from the first organic mixture;
(a4) concentrating the first organic mixture;
(a5) adding ethanol to the concentrated first organic mixture; and
(a6) adding water to the concentrated first organic mixture to precipitate the compound of Formula I in a yield of at least 75% and a purity of at least 98%.

27. The method of claim 1, wherein the compound of Formula I contains less than 1% of the compound of Formula X-5:

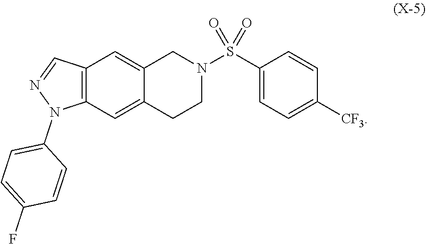 [SMILES: O=S(=O)(c1ccc(C(F)(F)F)cc1)N1CCc2cc3c(cnn3-c3ccc(F)cc3)cc2C1]

28. A method of preparing a compound of Formula IIa: 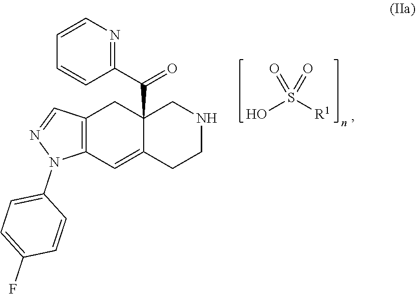
comprising:
(b) forming a second reaction mixture comprising a compound of Formula IIb: 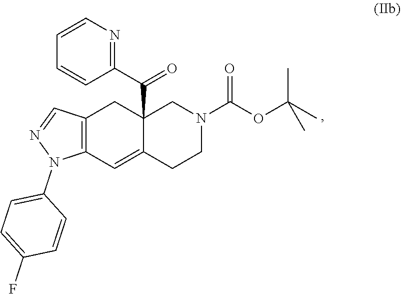 [SMILES: CC(C)(C)OC(=O)N1CCC2=Cc3c(cnn3-c3ccc(F)cc3)C[C@]2(C(=O)c2ccccn2)C1]
and
a sulfonic acid of the formula: 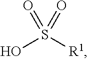
to form the compound of Formula IIa,
wherein
R¹ is C₁₋₆ alkyl, C₁₋₁₀ haloalkyl, phenyl, or 4-methylphenyl; and
subscript n is 1 to 4.

29.-41. (canceled)

42. A method of preparing a compound of Formula I: 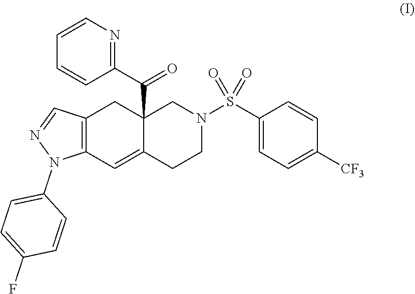 [SMILES: O=C(c1ccccn1)[C@]12Cc3cnn(-c4ccc(F)cc4)c3C=C1CCN(S(=O)(=O)c1ccc(C(F)(F)F)cc1)C2]
or a pharmaceutically acceptable salt thereof, comprising:
(c) forming a third reaction mixture comprising tetrahydrofuran, toluene, iPrMgCl, a compound of Formula III: 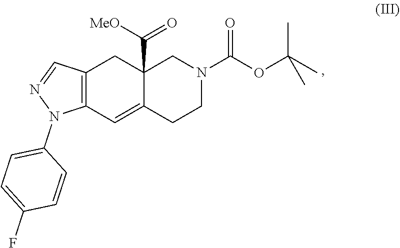 [SMILES: COC(=O)[C@]12Cc3cnn(-c4ccc(F)cc4)c3C=C1CCN(C(=O)OC(C)(C)C)C2]
2-bromo-pyridine: 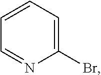
wherein the pyridine is present in a molar ratio of about 3.0 to the compound of Formula III, and wherein the Grignard reagent is present in a molar ratio of about 3.0 to the compound of Formula III;
(c1) adding acetic acid and water to the third reaction mixture to form a workup mixture;
(c2) distilling the workup mixture to form an intermediate mixture comprising a compound of Formula IIb: 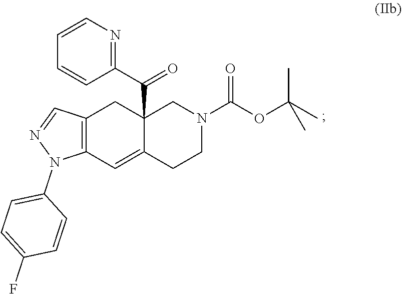 [SMILES: CC(C)(C)OC(=O)N1CCC2=Cc3c(cnn3-c3ccc(F)cc3)C[C@]2(C(=O)c2ccccn2)C1]
(b) forming a second reaction mixture comprising the intermediate mixture, acetonitrile, and methanesulfonic acid, to form a compound of Formula IIa having the structure: 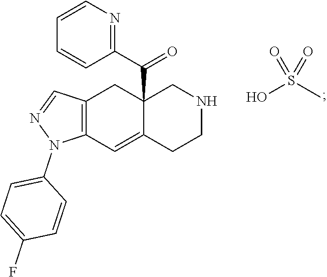
(a) forming a first reaction mixture comprising the compound of Formula IIa, triethylamine, ethyl acetate, and 4-(trifluoromethyl)benzenesulfonyl chloride: 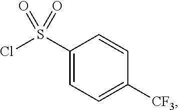 [SMILES: O=S(=O)(Cl)c1ccc(C(F)(F)F)cc1]
wherein the sulfonyl chloride is present in a molar ratio of about 1.0 to the compound of Formula IIa;
(a1) adding N-methylpiperazine to the first reaction mixture to remove unreacted 4-(trifluoromethyl)benzenesulfonyl chloride;
(a2) adding HCl and water to the first reaction mixture to partition the first reaction mixture into a first water mixture and a first organic mixture;
(a3) separating the first water mixture from the first organic mixture;
(a4) concentrating the first organic mixture;
(a5) adding ethanol to the concentrated first organic mixture; and
(a6) adding water to the concentrated first organic mixture to precipitate the compound of Formula I in a yield of at least 75% and a purity of at least 98%.

43. A method of preparing a compound of Formula IIb: 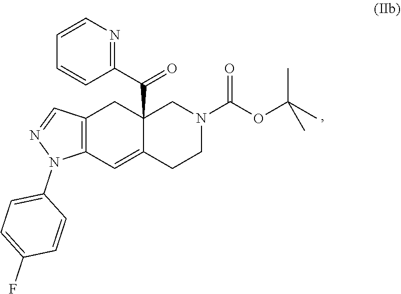 [SMILES: CC(C)(C)OC(=O)N1CCC2=Cc3c(cnn3-c3ccc(F)cc3)C[C@]2(C(=O)c2ccccn2)C1]
comprising the steps of:
(c) forming a third reaction mixture comprising a Grignard reagent, a compound of Formula III: 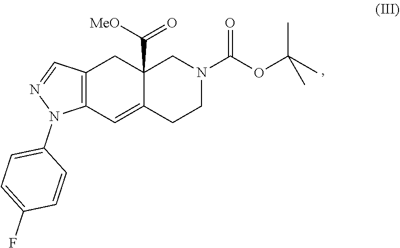 [SMILES: COC(=O)[C@]12Cc3cnn(-c4ccc(F)cc4)c3C=C1CCN(C(=O)OC(C)(C)C)C2]
and
2-bromo-pyridine: 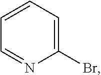
wherein the pyridine is present in a molar ratio of 2.8 to 3.2 to the compound of Formula III, and wherein the Grignard reagent is present in a molar ratio of 2.8 to 3.3 to the compound of Formula III, to prepare the compound of Formula IIb.

44.-50. (canceled)

51. A composition comprising:
a compound of Formula I in an amount of at least 99%: 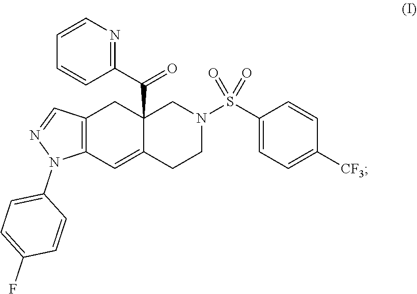 [SMILES: O=C(c1ccccn1)[C@]12Cc3cnn(-c4ccc(F)cc4)c3C=C1CCN(S(=O)(=O)c1ccc(C(F)(F)F)cc1)C2]
and
one or more impurity in an amount of from 0.01 to 1%.

52.-55. (canceled)

56. A crystalline form of (R)-(1-(4-fluorophenyl)-1,4,5,6,7,8-hexahydro-4aH-pyrazolo[3,4-g]isoquinolin-4a-yl)(pyridin-2-yl)methanone that is:
(R)-(1-(4-fluorophenyl)-1,4,5,6,7,8-hexahydro-4aH-pyrazolo[3,4-g]isoquinolin-4a-yl)(pyridin-2-yl)methanone methanesulfonic acid;
(R)-(1-(4-fluorophenyl)-1,4,5,6,7,8-hexahydro-4aH-pyrazolo[3,4-g]isoquinolin-4a-yl)(pyridin-2-yl)methanone camphorsulfonic acid;
(R)-(1-(4-fluorophenyl)-1,4,5,6,7,8-hexahydro-4aH-pyrazolo[3,4-g]isoquinolin-4a-yl)(pyridin-2-yl)methanone dibenzoyl-L-tartaric acid; or
(R)-(1-(4-fluorophenyl)-1,4,5,6,7,8-hexahydro-4aH-pyrazolo[3,4-g]isoquinolin-4a-yl)(pyridin-2-yl)methanone 4-nitrobenzene acid.

57. A crystalline form of (R)-(1-(4-fluorophenyl)-1,4,5,6,7,8-hexahydro-4aH-pyrazolo[3,4-g]isoquinolin-4a-yl)(pyridin-2-yl)methanone methanesulfonic acid: 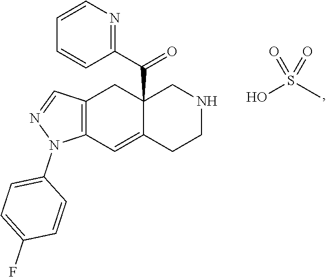
characterized by an X-ray powder diffraction (XRPD pattern having peaks at about 7.8, 14.7, and 15.5° 2-θ±0.2° 2-θ.

58.-64. (canceled)

65. A pharmaceutical composition comprising a composition of claim 1, and one or more pharmaceutically acceptable excipients.

66. (canceled)

67. A method of treating a disorder or condition through modulating a glucocorticoid receptor, comprising administering to a subject in need of such treatment, a therapeutically effective amount of a composition of claim 51, thereby treating the disorder or condition.

68. A method of treating a disorder or condition through antagonizing a glucocorticoid receptor, comprising administering to a subject in need of such treatment, a therapeutically effective amount of a composition of claim 51, thereby treating the disorder or condition.

69. A method of treating amyotrophic lateral sclerosis (ALS), comprising administering to a subject in need thereof, a therapeutically effective amount of a composition of claim 51, thereby treating ALS.
